(19) United States
(12) Patent Application Publication
(10) Pub. No.: US 2026/0069756 A1
(43) Pub. Date: Mar. 12, 2026

(54) SYSTEMS AND METHODS FOR MONITORING THE FUNCTIONALITY OF A BLOOD VESSEL

(71) Applicant: PatenSee Ltd., Or Yehuda (IL)

(72) Inventors: Gal GOSHEN, ModiIn (IL); Oz Moshe SEADIA, Bat Yam (IL); Hagay DRORI, Tel-Aviv (IL)

(73) Assignee: PatenSee Ltd., Or Yehuda (IL)

(21) Appl. No.: 19/284,894

(22) Filed: Jul. 30, 2025

Related U.S. Application Data
(63) Continuation of application No. PCT/IL2024/050115, filed on Jan. 30, 2024.
(60) Provisional application No. 63/441,892, filed on Jan. 30, 2023.

(51) Int. Cl. A61M 1/36

(52) U.S. Cl. CPC A61M 1/3656; A61M 2205/3306; A61M 2205/502; A61M 2205/587

(57) ABSTRACT

A method for displaying data on a patient's arm, the method including capturing an image of a patient's arm, analyzing the image, thereby obtaining information regarding a blood vessel in the patient's arm, displaying the information by projecting a light pattern on the patient's arm. A system for displaying data on a patient's arm, the system including a light source for illuminating a patient's arm, a sensor for capturing an image of the patient's arm, a computer for analyzing the image, thereby obtaining information regarding blood vessel in the patient's arm, a projector for displaying the information by projecting a light pattern on the patient's arm. Related apparatus and methods are also described.

### RELATED APPLICATIONS

This application is a Continuation of PCT Patent Application No. PCT/IL2024/050115 having international filing date of Jan. 30, 2024, which claims the benefit of priority under 35 USC § 119(e) of U.S. Provisional Patent Application No. 63/441,892 filed on Jan. 30, 2023. The contents of the above applications are all incorporated by reference as if fully set forth herein in their entirety.

### FIELD AND BACKGROUND OF THE INVENTION

The invention relates generally to the field of monitoring blood vessels in patients. Some aspects relate more particularly to early diagnosis of failure in blood vessel functionality, and even more particularly to early detection of failure of vascular access in patients undergoing hemodialysis treatments. Some aspects relate more particularly to measurements of fistulas.

Dialysis vascular access (VA, fistula or graft) makes life-saving hemodialysis treatments possible but also prone to access related problems.

The term “VA”, or “vascular access”, in all its grammatical forms, is used throughout the present specification and claims to mean all types of vascular access constructs, biological as well as synthetic, including, by way of some non-limiting examples, an arteriovenous fistula (AV), a synthetic graft, and an intravenous catheter.

One type of long-term access is an AV fistula. A surgeon connects an artery to a vein, usually in an arm or leg, to create an AV fistula. When the surgeon connects the artery to the vein, the vein grows wider and thicker, making it easier to place needles for dialysis. The AV fistula also has a large diameter that allows blood to flow out and back into a body quickly. A goal of an AV fistula is to allow high blood flow so that a large amount of blood can pass through a dialyzer.

VA function and patency are essential for optimal management of HD patients. Low VA flow and loss of patency limit hemodialysis delivery, extend treatment times, and may result in under-dialysis that leads to increased morbidity and mortality. In long-term VAs, especially grafts, thrombosis is the leading cause of loss of VA patency and increases healthcare expenditure.

A basic concept for VA monitoring and surveillance is that progressive stenoses develop over variable intervals in the great majority of VAs and, if detected and corrected (corrective procedure such as percutaneous transluminal angioplasty-PTA), under-dialysis can be minimized or avoided (dialysis dose protection) and the rate of thrombosis can be reduced. A number of monitoring and surveillance methods are available: sequential VA flow, sequential dynamic or static pressures, recirculation measurements, and physical examination.

Monitoring is the examination and evaluation of the VA to diagnose VA dysfunction using physical examination, usually within the HD unit, in order to detect the presence of dysfunction and correctable lesions before VA loss.

Physical examination can be used as a monitoring tool to exclude low flow associated with impending fistula and graft failures. Typically, there are 3 components to the VA examination: inspection, palpation, and auscultation.

A simple inspection can reveal the presence of swelling, ischemic fingers, aneurysms, and rich collateral veins. A strong pulse and weak thrill in the vein central to the anastomosis indicates a draining vein stenosis. Strictures can be palpated, and the intensity and character of the bruits can suggest the location of stenoses. A local intensification of bruit over the graft or the venous anastomosis compared with the adjacent segment suggests a stricture or stenosis. Physical examination can also include the elevation test, which consists of the elevation of the extremity with the VA and examination of the normal collapse of the access. The test is considered normal when the fistula collapses after the organ is elevated above the heart level of the patient.

Additional background art includes:
U. S. Patent Application Publication No, 2021/0015991 of Drori et al.

The disclosures of all references mentioned above and throughout the present specification, as well as the disclosures of all references mentioned in those references, are hereby incorporated herein by reference.

### SUMMARY OF THE INVENTION

The invention relates generally to diagnosis of failure in blood vessel functionality, and even more particularly to early detection of failure of vascular access in patients undergoing hemodialysis treatments.

According to an aspect of some embodiments of the present disclosure there is provided a method for displaying data on a patient's arm, the method including capturing an image of a patient's arm, analyzing the image, thereby obtaining information regarding a blood vessel in the patient's arm, displaying the information by projecting a light pattern on the patient's arm.

According to some embodiments of the disclosure, the projecting includes projecting using a Digital Light Processing (DLP) projector.

According to some embodiments of the disclosure, the information includes a mapping of skin vibrations on the patient's arm.

According to some embodiments of the disclosure, the information includes a mapping of blood vessels in the patient's arm.

According to some embodiments of the disclosure, the information includes a mapping of pulsatility in the patient's arm.

According to some embodiments of the disclosure, the information includes a suggested location for inflow needle insertion point in the patient's arm.

According to some embodiments of the disclosure, the information includes a suggested location for outflow needle insertion point in the patient's arm.

According to some embodiments of the disclosure, the information includes a location where it suggested not to insert a needle.

According to some embodiments of the disclosure, the information includes instructions for treatment.

According to some embodiments of the disclosure, the information includes numerical data produced as a result of the analysis.

According to some embodiments of the disclosure, the information includes a needling plan.

According to some embodiments of the disclosure, the needling plan is based on analyzing more than one image, at least two of the images used for the analyzing captured at different times.

According to some embodiments of the disclosure, the needling plan is based on historic information on previous insertion points to suggest locations for inflow and outflow needle insertion points.

According to some embodiments of the disclosure, the capturing the image of the patient's arm includes capturing a plurality of images of the patient's arm, and the analyzing the image includes analyzing the plurality of images.

According to some embodiments of the disclosure, the method further comprises displaying said captured image, including said information, on a display.

According to an aspect of some embodiments of the present disclosure there is provided a system for displaying data on a patient's arm, the system including a light source for illuminating a patient's arm, a sensor for capturing an image of the patient's arm, a computer for analyzing the image, thereby obtaining information regarding blood vessel in the patient's arm, a projector for displaying the information by projecting a light pattern on the patient's arm.

According to some embodiments of the disclosure, the light source includes a laser illuminator arranged to illuminate an area on the patient's arm.

According to some embodiments of the disclosure, the light source includes a laser illuminator arranged to illuminate an area on the patient's arm with a pattern of spots.

According to some embodiments of the disclosure, the light source includes a source of structured light.

According to some embodiments of the disclosure, the projector includes a source of structured light.

According to some embodiments of the disclosure, the projector includes a Digital Light Processing (DLP) projector.

According to some embodiments of the disclosure, the source of structured light includes a same DLP projector as the projector.

According to an aspect of some embodiments of the present disclosure there is provided a method for mapping areas of vibration on a patient's arm, the method including illuminating a patient's arm, capturing an image of the patient's arm, analyzing the image, thereby obtaining information regarding vibration on a skin of the patient's arm, and displaying a mapping of areas of vibration on the patient's arm based on the analyzing.

According to some embodiments of the disclosure, the illuminating includes illuminating using coherent light.

According to some embodiments of the disclosure, the illuminating includes illuminating an area on the patient's arm.

According to some embodiments of the disclosure, the illuminating includes illuminating using structured light.

According to some embodiments of the disclosure, the illuminating includes illuminating with a Digital Light Processing (DLP) projector.

According to some embodiments of the disclosure, the information includes a mapping of skin vibrations on the patient's arm.

According to some embodiments of the disclosure, further including displaying the information by projecting a light pattern on the patient's arm.

According to some embodiments of the disclosure, the displaying the information includes displaying a mapping of the information regarding vibration on the skin of the patient's arm.

According to some embodiments of the disclosure, the displaying the mapping includes displaying where the vibration on the skin of the patient's arm exceeds a threshold level of vibration.

According to some embodiments of the disclosure, wherein the displaying the mapping includes displaying the mapping registered to the patient's arm.

According to some embodiments of the disclosure, the capturing the image of the patient's arm includes capturing a plurality of images of the patient's arm, and the analyzing the image includes analyzing the plurality of images.

According to an aspect of some embodiments of the present disclosure there is provided a system for mapping areas of vibration on a patient's arm, the system including a light source for illuminating a patient's arm, and a sensor for capturing an image of the patient's arm, a computer for analyzing the image, thereby obtaining information regarding vibration on the patient's arm.

According to some embodiments of the disclosure, the light source includes a laser illuminator arranged to illuminate an area on the patient's arm.

According to some embodiments of the disclosure, the light source includes a laser illuminator arranged to illuminate an area on the patient's arm with a pattern of spots.

According to some embodiments of the disclosure, the light source includes a source of structured light.

According to some embodiments of the disclosure, including a projector for displaying the information by projecting a light pattern on the patient's arm.

According to some embodiments of the disclosure, the projector includes a source of structured light.

According to some embodiments of the disclosure, the projector includes a Digital Light Processing (DLP) projector.

According to some embodiments of the disclosure, the source of structured light includes a same DLP projector as the projector.

According to an aspect of some embodiments of the present disclosure there is provided a method for displaying a needling plan on a patient's arm, the method including providing historic information regarding needle insertion points on a patient's arm, capturing an image of the patient's arm, analyzing the image and the historic information, thereby obtaining information regarding a blood vessel in the patient's arm, producing a needling plan, and displaying the needling plan by projecting a light pattern on the patient's arm.

According to some embodiments of the disclosure, the historic information includes images of the patient's arm taken prior to the capturing the image of the patient's arm.

According to some embodiments of the disclosure, the historic information includes images of the patient's arm taken more than one day prior to the capturing the image of the patient's arm.

According to an aspect of some embodiments of the present disclosure there is provided a method for displaying where not to insert a needle on a patient's arm, the method including providing historic information regarding needle insertion points on a patient's arm, capturing an image of the patient's arm, analyzing the image and the historic information, thereby obtaining derived information regarding a blood vessel in the patient's arm, based on the derived information, determining where on the patient's arm it is not advisable to insert a needle, and displaying where on the patient's arm it is not advisable to insert a needle.

According to some embodiments of the disclosure, the displaying where on the patient's arm it is not advisable to insert a needle includes by projecting a light pattern on the patient's arm.

According to some embodiments of the disclosure, the system further comprises a display configured for displaying said captured image including said obtained information.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as monitoring blood vessels in patients, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified illustration of a system for measuring blood vessels according to an example embodiment of the invention;

FIG. 2 is a simplified block diagram of a system for measuring blood vessels according to an example embodiment of the invention;

FIG. 3 is a simplified block diagram of a system for measuring blood vessels according to an example embodiment of the invention;

FIG. 4 is a table showing a procedure for a medical person to examine a patient with reference to vascular stenotic lesions or thrombosis;

FIG. 5 is a simplified flow chart illustration of a method of examining a patient according to an example embodiment of the invention;

FIG. 6 is a simplified flow chart illustration of a method for transforming data from a stream of images to a frequency spectrum according to an example embodiment of the invention;

FIG. 7 is a graph showing power spectrum of vibrations measured by analysis of images produced by laser speckle imaging;

FIG. 8 is a simplified illustration of a system for measuring blood vessels according to an example embodiment of the invention;

FIG. 9 is a simplified illustration of optional lighting modes and optional images captured according to an example embodiment of the invention;

FIG. 10 is a simplified illustration of laser vibrometry employed to “listen” to a fistula according to an example embodiment of the invention;

FIG. 11 is a simplified illustration of laser speckle vibrometry employed to assess vibrations according to an example embodiment of the invention;

FIG. 12 is a simplified illustration of various projections onto a patient's arm according to an example embodiment of the invention;

FIG. 13 is a graph which shows a Receiver Operator Characteristics (ROC) curve of a stenosis detecting model according to an example embodiment.

FIG. 14 is a simplified flow chart illustration of a method for displaying data on a patient's arm according to an example embodiment of the invention;

FIG. 15 is a simplified flow chart illustration of a method for mapping areas of vibration on a patient's arm according to an example embodiment of the invention;

FIG. 16 is a simplified flow chart illustration of a method for displaying a needling plan on a patient's arm according to an example embodiment of the invention; and

FIG. 17 is a simplified flow chart illustration of a method for displaying where not to insert a needle on a patient's arm according to an example embodiment of the invention.

### DESCRIPTION OF SPECIFIC EMBODIMENTS OF THE INVENTION

The invention relates generally to the field of monitoring blood vessels in patients. Some aspects relate more particularly to early diagnosis of failure in blood vessel functionality, and even more particularly to early detection of failure of vascular access in patients undergoing hemodialysis treatments. Some aspects relate more particularly to measurements of fistulas.

### Introduction

Monitoring by physical examination is cost-effective and a proven method to detect VA abnormalities. Unfortunately, nephrologists and HD staff generally have limited availability and are not well informed. As a result, regular physical examinations of VAs are not generally carried out in HD units.

Thus, there is a need for monitoring solutions capable of detecting a forming stenosis early and predicting thrombosis, which overcome at least some of the following drawbacks of existing monitoring practices:
Poor compliance to routine VA physical examination by dialysis centers as outlined by guidelines;
An inherent inaccuracy related to a single-time physical examination or pressure/flow measurement of the VA;
An inherent inaccuracy of a single parameter such as flow or pressure;
Periodic measurements' results may be influenced by unrelated hemodynamic events; and
Measurement by different human caretakers may introduce inconsistencies.

An aspect of some embodiments of the present invention relates to replacing or adding to physical examination performed by medical staff/nurses.

When a nurse or physician examines a patient's blood vessels, they typically use a three-step procedure: look, listen and feel.

An aspect of some embodiments is related to performing look, listen and feel by instruments measurements and computerized analysis.

In some embodiments, systems as described herein perform a look, listen and feel based on illuminating and imaging a patient's limb and analyzing the data collected from the imaging. In some embodiments, the systems teach how to predict fistula condition and potentially enable early prevention of failure.

In some embodiments, methods as described herein performs a look, listen and feel based on illuminating and imaging a patient's limb and analyzing the data collected from the imaging. In some embodiments, the systems teach how to predict fistula condition and potentially enable early prevention of failure.

In some embodiments blood flow is measured in a non-invasive manner, based on image processing of image of blood vessels in a human body. Physiological parameters which are known to affect vascular access (VA) are measured, and the measurements are optionally used to determine whether a patient should be scheduled for corrective procedure or proceed to undergo dialysis.

An aspect of some embodiments is related to performing feel, as described herein, by instruments measurements and computerized analysis.

In some embodiments, the listen as described herein is performed by instruments, optionally the same instruments.

In some embodiments, the look as described herein is performed by instruments, optionally the same instruments.

An aspect of some embodiments of the present invention relates to automatic detection and/or monitoring of an AV fistula in an images of blood vessels.

In some embodiments an image of blood vessels is analyzed, and a location where an artery is connected to a vein is optionally determined to be a location of an AV fistula.

In some embodiments an image of blood vessels is analyzed, and a location where an artery appears to be connected to a vein is optionally determined to be a location of an AV fistula.

In some embodiments an image of blood vessels is analyzed, and an AV fistula is optionally measured to estimate geometric properties.

An aspect of some embodiments of the present invention relates to automatic, non-invasive measurement of parameters associated with blood flow.

In some embodiments the non-invasive measurement includes imaging blood vessels through skin, using reflected light and/or transmitted light.

In some embodiments, a probability of failure of vascular access is optionally estimated. In some embodiments the estimation is based on one or more of the parameters measured.

In some embodiments, a probability of occlusion formation is optionally estimated. In some embodiments the estimation is based on one or more of the parameters measured.

In some embodiments, a probability of thrombus formation is optionally estimated. In some embodiments the estimation is based on one or more of the parameters measured.

In some embodiments, a grade of stenosis is optionally estimated. In some embodiments the estimation is based on one or more of the parameters measured.

In some embodiments, a rate of stenosis formation is optionally estimated. In some embodiments the estimation is based on one or more of the parameters measured.

In some embodiments, a grade of VA maturation is optionally estimated. In some embodiments the estimation is based on one or more of the parameters measured.

In some embodiments, a rate of VA maturation is optionally estimated. In some embodiments the estimation is based on one or more of the parameters measured.

An aspect of some embodiments of the present invention relates to providing a visual report to a caregiver.

Attributes, one or more of which are related to some embodiments the invention, are listed below:
1. One or more of patient-related parameters, including images, are readily available for measurement(s) in a way that is potentially cost-effective and/or non-invasive (optionally, non-contact), and/or integrated into routine dialysis appointments.
2. An input to an algorithm described herein optionally includes one or more patient-related parameters in order to estimate probability of failure of vascular access, where each of the parameters can be available on a single-measurement basis or as multiple measurements along the time axis.
3. Some of the patient-related parameters are obtained using objective measurements, potentially not requiring high competence from a user, such as a patient and/or a health care professional.
4. Some of the patient-related parameters are optionally taken from the patient's specific medical record and include elements such as demographics (e.g., age, gender, weight and height), lab tests, imaging tests (e.g. X-ray, MRI) and results of a physical exam. It should be clear to a person skilled in the art that the parameters can be extracted in multiple ways, for example—directly typing exam results into a keyboard connected to a system as described herein, a computer process that accesses electronic medical records using a specific patient ID, speech-to-text conversion, voice recognition algorithms applied to verbal analysis of the staff and OCR of a printed/written documents.

Measurement of VA maturation: The VA has a unique tissue structure when compared with veins and arteries. The structure changes during a VA maturation process, and during a stenotic process.

Structural changes impact the mechanical and optical characteristics of the VA, thus monitoring of changes can potentially be measured, in some embodiments, by one or more of:
Imaging: by way of a non-limiting example by measuring changes in contrast or intensity of reflected light and/or transmitted light;
Non imaging: intensity of reflected light or transmitted light;
Measurement of scattering and absorption coefficients (e.g., two distance steady state photon migration measurement).

For example, in some embodiments a system is configured to detect veins, monitoring VA during a maturation period potentially alters detection results. In an example of optical sensing, the response of the VA to light (one or more of transmission, reflection, absorption, scattering) potentially changes over the maturation period. Monitoring of maturation is potentially beneficial to raise a success rate of VA maturation by suggesting a timely pre-emptive correction. Measurement of blood vessel layers, or a ratio between blood vessel layers or the changes in ratio between layers or changes in absolute values of layers during maturation or stenotic process.

Accuracy of estimating maturation (maturity level, stage, rate, completion) or probability of failure of vascular access, occlusion formation and probability of thrombus may be improved by using one or more parameters generated from non-invasive measurement. The parameters used can be directly measured or be a result of a pre-processing applied on the measurement. Such pre-processing can be application of various algorithms as well as combination of several parameters and utilization of multiple measurements over time.

Examples for metrics or phenomena that are optionally extracted and used in some embodiments of the invention:

1 Pulse wave velocity—In some embodiments detect reflection or absorption of optical radiation from at least two points in an image frame. In some embodiments changes in electrical impedance as measured by electrodes placed between and/or along the two points, along the blood vessel or tissue area. Optionally, the two points include sections known to be more susceptible to develop stenosis. More generally, at least one point is used for measuring pulse wave shape (such as, for example, pulse wave amplitude, Full-Width Half Max (FWHM)).

In some embodiments pulse wave amplitude is optionally measured. An optional method for measuring pulse wave amplitude includes measuring a first measurement of an area of a location along a vein identified as a widening of a blood vessel due to a pulse wave. An area of the same location in a different image, when the pulse wave is not at that location, is also measured in a second measurement. A difference between the first measurement and the second measurement is optionally associated with the pulse wave amplitude. In some embodiments the pulse wave amplitude is taken as a feature which corresponds to mechanical properties of a vein all, and/or with maturity of an AV through which the pulse wave travels.

In some embodiments a Pulse Wave Analysis (PWA) is optionally performed to assess variance related to vascular stiffness which is associated with additional risk factors such as cardiovascular disease or atherosclerosis which in turn—may impact viability over time of the VA. A quality of the pulse is optionally scored, and changes over time and between different sections are optionally included in the analysis, in some embodiments.

In some embodiments, a Field of View (FoV) of a camera is arranged to be equal to or larger than a distance which a pressure pulse travels during a time span between consecutive captured images.

In some embodiments, an illuminated area upon a patient's arm is arranged to be equal to or larger than a distance which a pressure pulse travels during a time span between consecutive captured images.

2. Appearance and development of collateral veins and their characteristics, such as: density, size, distance from the VA, orientation, filling etc. by image processing and/or other detection methods, e.g., measure contrast—by absorption of light in the visible or NIR wavelength; or emission at the far IR wavelength. Other measurement options include measuring an amount of change of absorption in the visible and near IR and amount of emission in the far IR. Another optional way to measure development of collaterals is optionally measuring temperature changes of the VA surrounding. In some embodiments detection of appearance and development of collateral veins optionally uses reference images or measurements taken from a prior examination. In some embodiments trend analysis of collateral vein development rate optionally uses frequent examinations. The examinations are optionally performed daily, every dialysis session, every week, bi-weekly, or monthly.

In some embodiments collateral veins are detected by comparing a new image to a previous image and counting veins—an increase in the number of veins is optionally taken to mean that the new veins are collateral veins.

In some embodiments, appearance and/or development of collateral veins is detected by extracting features from one image or measurement.

Rationale: detection of a collateral vessel potentially indicates a flow limiting (hemodynamic significant) lesion. Collateral vessels may develop and enlarge, dissipating the increased intra-access pressures in the setting of outflow stenosis.

3. A blood vessel's smallest diameter by image processing (stenosis location).

4. Detection of a point of narrowing by estimation of mechanical reflection waves or changes in local pressure/flow for example by measurement of electrical impedance changes.

5. A blood vessel's largest diameter by image processing (appearance and size of aneurysms).

6. Detection of vessel collapse when arm or leg is elevated.

7. Using Near Infrared (NIR) (700-1000 nm) reflected and/or transferred spectroscopy for measuring amounts of oxygenated and deoxygenated hemoglobin (Hb).

8. Spectroscopy analysis for oxygenated and deoxygenated hemoglobin (Hb) 9. Audible sound of the VA (bruit).

10. Palpated pulsation of the VA (thrill).

11. Analysis of electrical impedance changes at VA using signal processing methods known in the art.

12. In some embodiments in which multiple measurements of the same parameter are taken over time, the measurements may be synched according to a detected breathing cycle and categorized for the detection algorithm in respect to their relative time along the breathing cycle. Such synching and categorization are potentially beneficial, for example, when evaluating changes in the oxygen mix over time, but can also improve accuracy of other measurements, such as pulse wave velocity.

Output of a system as described herein may be in the form of an audible alarm, visual alarm, image, sequence of images, or a video providing the medical personnel guidance for fast and accurate intervention (e.g., give a recommendation to the medical personnel regarding the best location(s) for intervention). The system may recommend treatment for a patient (PTA, not to intervene, thrombectomy). The recommendation is optionally based on information collected by the system.

According to an aspect of some embodiments of the present invention, output of the system during a test is optionally analyzed and/or optionally used to guide a patient through a test in order to perform the test correctly. By way of a non-limiting example, in an elevation test-verifying that the elevation/position of a limb is correct. In some embodiments, there is also an alert to a nurse/technician in case a patient has not performed the test correctly or requires help.

In some embodiments, the above-mentioned output is optionally used to support a remote physical examination to be performed by a patient while the system provides feedback on correct performance of the examination and/or alerts remote support personnel, such as a nurse or technician that additional guidance is required.

In some embodiments, system output is optionally provided differently to different consumers of the data. For example: a dialysis nurse is optionally provided with a general interpretation on a likelihood of clinically meaningful stenosis formation and an interventional radiologist is optionally provided with an alert with an annotated image and/or optionally a report highlighting parameters such as location, severity and rate of stenosis formation.

According to an aspect of some embodiments of the present invention there is provided a system and methods for measuring parameter related to fistulas.

In some embodiments, there is provided a system which includes optical apparatus to acquire one or more images of the same patient's fistula along a surveillance period.

In some embodiments, one or more measurements and/or features are optionally extracted from the image(s)—and their changes over time are optionally monitored. In some embodiments, the features are timeline derivatives of parameters measured or estimated in the image(s), by way of a non-limiting example changes in number, branching & size of collateral veins happening over a period of time, such as days/weeks/months.

In some embodiments, using a machine-learning-derived method to identify a pattern within the above changes which may potentially lead to a significant clinical end point (e.g., Stenosis of the fistula) before there are clinical signs or symptoms which human nurses can identify.

In some embodiments, a system is provided which measures parameters relating to a fistula by optical means.

In some embodiments, structured light is projected onto a patient's body or limb, and the body is imaged. In some embodiments, the structured light may include horizontal and/or vertical stripes of equal or different widths and/or various light patterns other than stripes.

In some embodiments, imaging the structured light is used to provide information about an extent of the fistula, for example length of a long axis of the fistula along the body: breadth of a short axis of the fistula along the body: shape of the fistula as it appears in the image: segmentation of the fistula circumference, eccentricity index and/or aspect ratio of each segment, smoothness and/or roughness of a fistula outline

In some embodiments, structured light patterns are projected onto a patient's body or limb, and the body is imaged, providing information about a three-dimensional shape of the fistula or organ.

In some embodiments, the system identifies changes in the shape of the fistula and/or an organ near the fistula. In some embodiments, a projector is used to project one or more light patterns (e.g., structured light). In some embodiments, a method measures and/or estimates how the patterns deform on a patient's organ to measure the organ's shape and shape changes over time.

In some embodiments, structured light patterns are projected onto a patient's body or limb, and the body is imaged, providing information about a three-dimensional shape of the fistula, by way of some non-limiting examples volume of an entire fistula or segments of a fistula (e.g. needle insertion points); characteristics and/or variance of curvature; changes in shape and/or volume of an underlying arm/organ section near a fistula; and three-dimensional surface features such as smoothness and/or roughness.

In some embodiments, Laser Speckle Interferometry (LSI) is used. In some embodiments, LSI is used to record and look at vibrations of the fistula surface that correlate with the blood flow and turbulence inside. Changes in the blood flow and turbulence are typically correlated with stenosis events, and potential development of clinical conditions.

In some embodiments, imaging the speckled light is used to provide information about dynamic effects in the fistula, for example heart pulse, blood flow turbulence, and optionally produce spectrograms of vibrations of a fistula.

In some embodiments, images of the body are taken some period of time apart, and differences between the images are optionally used to determine differences in the shape of the fistula.

In some embodiments, the images are taken days, weeks, months or years apart, and differences between the images is optionally used to measure and/or monitor changes in size or shape of the fistula.

In some embodiments, the images are taken seconds or minutes apart, for example with a limb such as a hand held horizontally followed by the hand held vertically, and differences between the images is optionally used to measure and/or monitor one or more of: whether at least some of the blood in the fistula can evacuate the fistula; a rate of blood evacuation; a degree of blood evacuation from the fistula and/or specific portions of the fistula; and collapse of one or more needle insertion points.

In some embodiments, the images are taken fractions of a second apart, as a video clip or movie, and differences between image frames is optionally used to measure and/or monitor dynamic parameter related to the fistula, such as heart pulse, blood flow turbulence, and optionally produce spectrograms of vibrations of a fistula.

In some embodiments, the spectrogram is optionally produced by selecting one or more pixels in the image frames which show a large or even a maximal variation of intensity over time. In some embodiments, the number of pixels selected is optionally in a range of 1-100 pixels. In some embodiments, the values of pixel intensity of this or these pixels are used to compute a function of light intensity over time. In some embodiments, a frequency spectrum of the light intensity is optionally produced by transforming from the time domain to the frequency domain, for example by a Fast Fourier Transform (FFT).

In some embodiments, an analysis is made of changes in the dynamic parameters relate to the fistula between imaging sessions, to monitor changes in the fistula and the patient's conditions.

In some embodiments, performing the above together with Near IR imaging potentially enables collecting data that correlates with examinations required to be perform by nurses and/or physicians and that is already clinically proven to have predictive value to identify stenosis events.

According to an aspect of some embodiments of the present invention there is provided a system and methods for implementing and recording more than one technique or modality, for example one or more of structured light; laser speckle interferometry; image analysis and Near IR imaging modalities, using one imaging device.

In some embodiments, the system includes a processor and an imaging device which includes a Digital Light Processing (DLP) projector and a Near IR camera.

According to an aspect of some embodiments of the present invention there are provided systems and methods for analyzing vibrations of light reflected from a patient's body.

In some embodiments, pulsatility of a heart is monitored.

In some embodiment, analyzing the pattern of vibrations caused by flow through or in vicinity to the fistula optionally detects full or partial occlusions of either the inflow or outflow pathways.

In some embodiment, analyzing the pattern of vibrations caused by flow through or in vicinity to the fistula while imposing local pressure to either inflow or outflow pathways optionally detects full or partial occlusions of either the inflow or outflow pathways.

In some embodiments, analyzing the vibrations optionally detects onset of flow through the fistula related to normal heart activity, (the diastole or systole phases of the heart cycle).

In some embodiments, analyzing the vibrations optionally detects onset of flow through the fistula related to sudden release, (partially or full collapse or expansion of the fistula).

In some embodiments, analyzing the vibrations optionally detects a period of inflow of blood to a fistula, followed by a sudden opening of an obstacle which enables blood to flow out of the fistula. Such opening may happen during high pressure of a heart systole. In some instances, the sudden opening is called hammering. In some embodiments, the hammering is detected by measuring amplitude of vibrations, optionally relative to the amplitude at other times, for example other times during a heartbeat.

In some embodiments, analyzing the vibrations related to onset of flow, for any or all types of onset, optionally measures a parameter value or a change in parameter value or a change in a characteristic parameter value, or a variance of the parameter value. The parameters may be one or more of: Intensity, Energy, Steepness of onset (derivative of value), Relaxation time, Temporal-width, Duty-cycle, Spectral-content, Spectral-width, or any combination of such.

In some embodiments, analyzing the vibrations related to onset of flow optionally measures a parameter value related to the time-delay or phase-delay between onsets related to sudden release, and onsets related to normal heart activity.

In some embodiments, analyzing the vibrations related to onset of flow optionally measures a parameter value related to the regularity or self-similarity of a series of onsets of the same source.

### Overview

An aspect of some embodiments of the present invention relates to providing a contactless, machine vision-based, monitoring system constructed by illumination with coherent light sources and a fast image sensor to capture imaging of a fistula's structure changes, sense sub-dermal information and read data related to blood vessel assessment for early detection of fistula stenosis. The present invention is designed to provide a way to streamline workflow in dialysis centers, to improve quality of dialysis patient care and to support caregiver staff. In some embodiments the system is optionally suitable for home use.

An aspect of some embodiments of the present invention relates to automatic, non-invasive collection of imaging information and analyzing the imaging information.

In some embodiments the imaging information is mapped, or registered, to blood vessel(s) and/or fistulas in a patient's organ being imaged. In some embodiments the patient's organ is the patient's arm.

In some embodiments information is optionally projected onto the patient's organ, for example on the patient's arm.

The projected information may be one or more of:
numerical data based on the analysis;
one or more correctly registered location(s) on the patient's organ, for example locations of a fistula, and/or a vibration map, and/or a pulsatility map, and/or a map showing results of analysis of one or more images of the patient's arm;
instruction(s) to a patient and/or a caregiver referring to treatment suggestions based on the analysis;
a needling plan. By way of a non-limiting example a needling plan based on information from different images taken at different time points, taking into account past information on previous insertion points to suggest location(s) for inflow and/or outflow needle insertion points;
information to support a specific needling method, such as rope ladder, buttonhole.

It is noted that historical data, that is documentation of previous needling locations, potentially enables producing a new needling plan. A good needling plan can potentially:
a. Minimize damage to the AV access during needling.
b. Minimize possible complications from needling.
c. Minimize pain and anxiety related to needling.

In some embodiments the system includes software to track a patient's arm in order to project information onto the patient's arm registered correctly to the patient's arm.

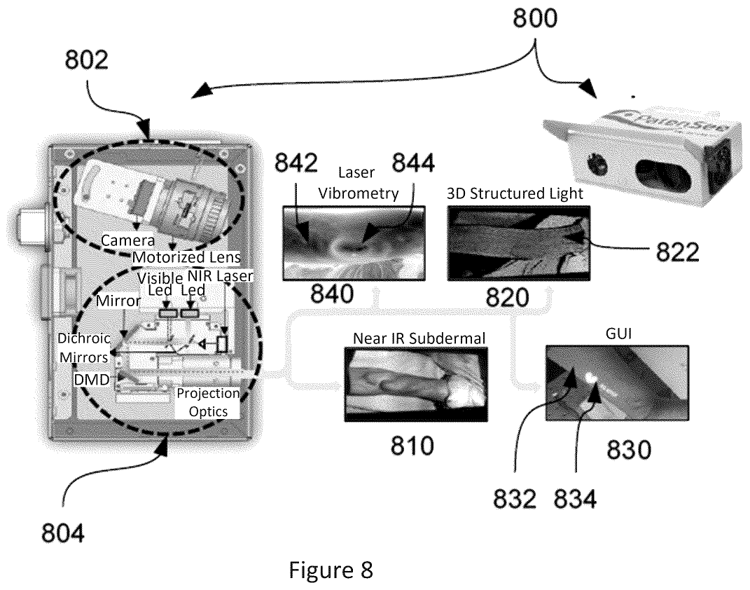

In some embodiments, not shown in FIG. 8, the detector(s) or camera(s) 802 and the illumination source(s) 804 optionally share a same line-of-sight toward the patient's arm, for example by both viewing the patient's arm, at least one of the detector(s) or camera(s) 802 and the illumination source(s) 804 through a semi-transparent mirror at 45 degrees to the line-of-sight.

The term “pulsatility” is used throughout the present specification and claims to mean values measured and/or calculated by the system and methods described herein. The values are based on one or more of vibrations; pulse wave shape properties; amplitude of a pulse wave; amplitude of vibrations; a slope of a pulse wave; a map of vibration; a map of differences in vibrations from previous measurements; a map of slopes in a 3D shape of the arm; a map of changes in the 3D shape of the arm.

The above-mentioned pulsatility values can be calculated based on image analysis, can optionally be registered to the patient's arm, and can optionally be projected onto the patient's arm.

An aspect of some embodiments of the present invention relates to displaying a needling plan on a patient's arm.

In some embodiments, historic information regarding previous needle insertion points on a patient's arm is used to provide the needling plan.

In some embodiments, an up-to-date image of the patient's arm is provided, or captured, and used to provide the needling plan.

In some embodiments, a vibration map is produced and used to provide the needling plan. In some embodiments the vibration map may be produced based on analysis of one or more image(s) of the patient's arm.

In some embodiments, the historic information optionally includes historic images of the patient's arm.

In some embodiments, the needling plan is based on analyzing one or more of the up-to-date image(s) and the historic image(s).

In some embodiments the historic images may be images taken more than one day prior to the analyzing a providing the needling plan.

In some embodiments the historic images may include prior images, taken at a previous needling treatment, even an immediately prior, unsuccessful needling attempt, up to prior images taken hours, days, weeks, or even months prior to capturing the image of the patient's arm.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

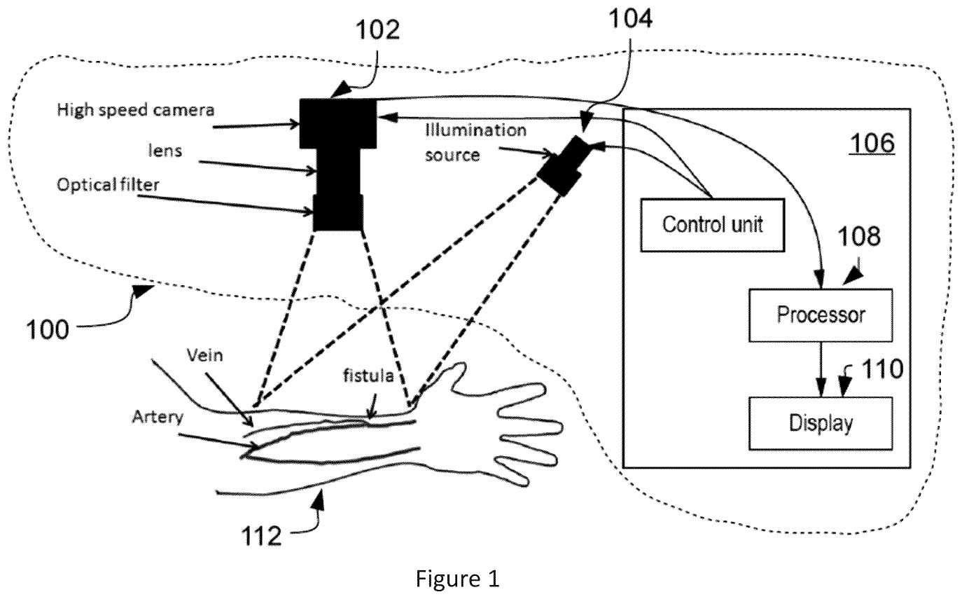

Reference is now made to FIG. 1, which is a simplified illustration of a system for measuring blood vessels according to an example embodiment of the invention.

FIG. 1 shows a top level set up configuration of an exemplary system 100 for measuring blood vessels.

In some embodiments the system 100 may include at least one illumination source 104 and at least one detector 102, such as a camera.

In some embodiments the system 100 may further include a control unit 106, which optionally activates the illumination source 104 and the camera 102, and an optional processor 108, which optionally receives and analyzes images generated by the camera 102.

In some embodiments, the generated images and/or the data generated following the analysis of the images may be displayed on an optional display 110 coupled to the processor 108, either wirelessly or via a wired connection.

In some embodiments, the processor 108 and the display 110 may be implemented in a single device, such as a laptop, tablet or smartphone. In some embodiments, a scan system may be applied that optionally moves the detection unit (automatically or manually) and optionally scans an organ at more than one point. FIG. 1 describes the system 100 applied to an arm 112.

The system and method are capable of implementation with other organs, without limitation.

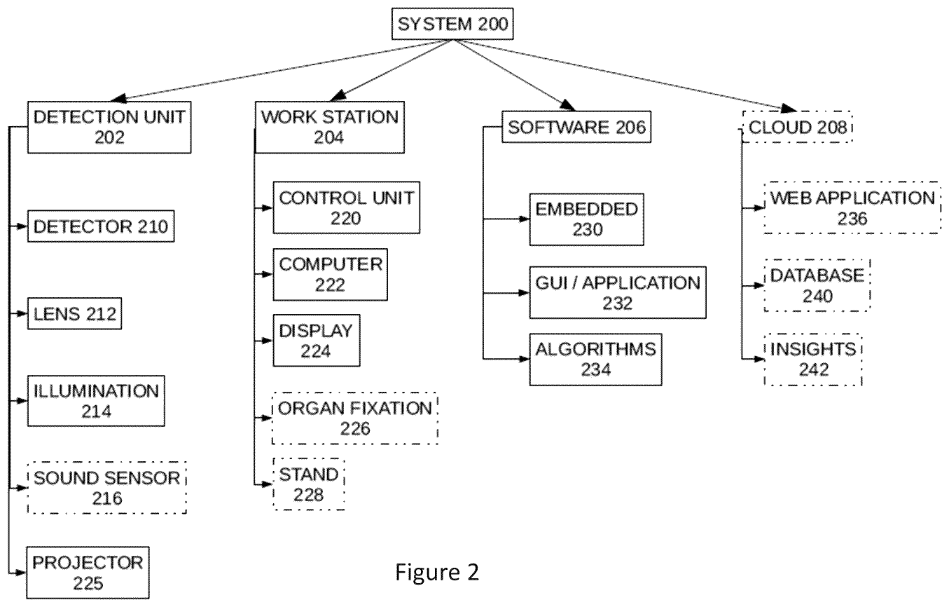

Reference is now made to FIG. 2, which is a simplified block diagram of a system for measuring blood vessels according to an example embodiment of the invention.

FIG. 2 describes the top-level block diagram of an exemplary system.

In some embodiments the system may include at least two main units; a detection unit 202 and a software unit 206.

The system may include additional units, such as a work station 204, optional cloud infrastructure 208, etc.

In some embodiments, the software unit 206 includes at least two sub-units, an embedded unit 230 and an algorithms unit 234. The software unit 206 may include additional blocks, such as a Graphical User Interface (GUI) unit 232, etc.

### Detection Unit

In some embodiments, the detection unit 202 optionally uses:
1. visual/optical detection, to acquire images containing information to be further analyzed.
2. Speckle imaging-When an object is illuminated by laser light, the backscattered light forms an interference pattern consisting of dark and bright areas. This pattern is called a speckle pattern. If the illuminated object is static, the speckle pattern is stationary. When there is movement in the object, such as red blood cells in a tissue, the speckle pattern will change over time. The speckled images contain information related to changes in the blood vessels which is optionally analyzed and extracted by image processing.
3. Dark field\side illumination—
   a. Specular reflections not reaching the camera
   b. Only diffused scattering rays are captured by the camera
   c. Reducing surface reflection
   d. Contrast profile changes with changing the angle between light source and detector.
4. Transmitted illumination-Illuminates the back surface of a sample. The sample is placed between the illumination source and the sensor device. Transmitted illumination potentially improves the image contrast and/or potentially increases the depth at which blood vessel can be imaged.
5. Photo acoustic imaging potentially enhances contrast between different mediums because of differences in changes in the optical characteristic of the different mediums. Photo acoustic imaging potentially reduces scattering in tissue because of averaging of the refraction index gradient in tissue components, potentially resulting in a greater penetration depth of light.

In some embodiments the detection unit 202 optionally includes one or more of the following components:
1. One or more detectors/sensors/cameras 210 (e.g., CCD or CMOS, InGaAs sensor, micro bolometer), which are sensitive to one or more of visible, near infrared light, short-wave infrared (SWIR) light. In some embodiments a sensor frame rate can range between single-frame to a high frame rate. Sensor frame rate are optionally in a range of, for example, 5, 10, 16, 24, 30, 50, 60, 100, 165, 200, and even up to 300-frames per second (fps).
2. One or more lenses 212 (zoom or fixed focal length) and/or filters
3. One or more illuminators 214 or emitters (e.g., an illumination source that can be coherent or non-coherent, narrow spectra or broadband, UV, visible, SWIR, far IR, NIR—for example NIR led or green (532 nm) laser). Emitters can be coaxial or in different angles relative to the detector 210 and a VA.

The operation mode can be stills or video.
4. One or more projectors 225, configured to project colored dots, lines, shapes, text, and such color markings onto a patient's arm.
5. One or more polarization filters (elliptical and/or linear)
6. One or more optical bandpass filters
7. The detection unit optionally includes a scan system or a moving bar scanner.

In some embodiments, the detection unit 202 optionally uses an audio/sound detection sensor 216, instead of, or in addition to, visual/optical detection, and the detection unit 202 may optionally include one or more audio sensors.

In some embodiments, the detection unit 202 may include vital signs sensors.

### Software Unit

In some embodiments, the software unit 206 may include one or more of the following components:
1. GUI—graphic user interface/Application 232 for one or more of: operating a test procedure, displaying images and/or results and/or inserting or importing patient clinical information, and/or controlling one or more projector(s) 225 to project colored dots, lines, shapes, text, and such color markings onto a patient's arm.
2. Embedded 230—for controlling the detection unit 202.
3. Algorithms unit 234—the algorithms unit optionally includes algorithms, or software modules, for: Image processing and/or Artificial Intelligence (AI), for example Machine learning (ML). The terms Artificial Intelligence, AI, Machine Learning, ML and any other similar terms known in the art are interchangeable.

In some embodiments, inputs for the ML algorithm are optionally images and/or data captured by the detection unit 202.

In some embodiments, the inputs may include also clinical information of the patient and/or vital signs.

In some embodiments, the work station 204 optionally includes a computer, a screen, a keyboard, one or more knob controls, a mechanical interface for the imaging unit, and an electric power supply or interface to electric power. In some embodiments, the work station 204 may also include an “organ fixation surface”.

In some embodiments, the work station 204 optionally includes one or more of: a control unit 220, for controlling operation of the detection unit 202 and/or one or more of the components of the detection unit 202;
a computer 220;
a display 224;
an optional organ fixation surface or device 226, for optionally placing an organ at a specific location relative to the illumination 214 and/or the detector 210; and
a stand 228, for placing components of the system at a specific location relative to a patient's organ.

In some embodiments, the cloud infrastructure 208 optionally includes one or more of the following cloud services
a storage (database) server 240;
a Web application server 236;
a computing service for machine learning, such as refining algorithm(s) based on new data; and/or for analytics—to provide measures of function and metrics to a user; and/or insight—to provide metrics related to current or a predicted future clinical condition of the VA.

A machine learning algorithm—may be supervised or unsupervised, learning based on database of images and/or of patient parameters produced by an embodiment of the invention, and/or of meta data such as a patient's, disease, vital signs, parameters from a dialysis machine and/or other data available in a medical electronic record, optionally including previous interventions for this patient, additional risk factors, comorbidities, and so on.

The steps include one or more of:
1. Feature extraction from the images
2. Trend calculation of the features
3. Running ML on the feature vectors and/or on the features vector trends.

In some embodiments an outcome of the ML is a statistical classifier model that distinguishes between less or more than 50% AV patency.

In some embodiments Analytics and Insight run on the metadata and patient records, and calculate statistics of failure of the AV based on the patient profile (metadata and medical health record).

In some embodiments analytics is optionally performed on a clinic's performance, for example how many stenosis events per year.

### Scaling Algorithm

In some embodiments, a scaling algorithm calculates the image scale (for example scaling pixels to mm). The scaling may be used for calculating absolute or relative values of one or more of a vessel's radius, pulse wave velocity, size of collateral vessels, density of collateral vessels, and distance of collateral vessel from VA.

### Registration Algorithm

In some embodiments, a registration algorithm may perform automatic or semi-automatic registration between two or more sequential images.

The registration algorithm may align and/or scale two or more images that contain the same object in different positions or angles of view or different fields of view.

In some embodiments inputs to the registration algorithm include at least two images and in case of semi-automatic registration, optionally, one or more points that are marked by the user on the two images.

The registration algorithm potentially enables the system to measure a variation between at least two examinations, no matter how the arm, or another examined organ, is positioned during the different examinations.

In some embodiments, registration of at least two images of the same patient that contain a VA object is optionally done by detection (e.g., segmentation) of the VA and fitting the VA image in a first image by geometrical transformation to the VA image in a second image.

### Detection of Vascular Access (VA) Body Algorithm

In some embodiments automatic or semi-automatic detection of the vascular access body location in the image is performed.

In some embodiments input for an algorithm for detection of a vascular access body includes at least one image that contains the vascular access body in the image frame.

In some embodiments an optional input is a set of one or more points along a blood vessel which includes the VA body, optionally marked by a physician/nurse on an image which includes the VA body.

The algorithm output may be a set of the vascular access body pixels in the image.

In some embodiments computerized detection of the VA body is based on a unique VA shape, size, orientation, position and etc.

In some embodiments, a device such as, by way of a non-limiting example, an “ELY-1000 vascular imaging instrument for Arterial puncture” as developed by ELYNNSH MEDICAL, is used. The device, according to the manufacturer, assists medical staff in identifying subcutaneous arteries during an arterial puncture, and can conveniently & quickly display the exact location of the arteries and direction.

In some embodiments a location is detected in an image, where an artery and a vein are connected or appear to join.

In some embodiments, a blood vessel providing blood to a VA is elevated by surgery toward the skin surface. Because of depth differences of blood vessel segments, an image which cover a field-of-view (FOV) which includes a VA, the VA often appears as a closed contour centroid. Tissues surrounding the VA body are often deeper under the skin than the VA body.

In some embodiments, the difference in depth is optionally detected by the VA body potentially showing up as a darker area than native or surrounding vessels. For example, when NIR illumination is used, the NIR light is absorb in the blood Hgb, and blood vessels closer to the surface appear darker than deeper vessels.

### Example Embodiment—System Description

The system may measure one or more of the following example phenomena: vessel diameter, pulse wave velocity, NIR (e.g., 700-1000 nm) reflected spectroscopy, appearance of collateral veins and their characteristics, such as: density, size, distance from the vascular access and oxygen concentration at the vascular access.

In some embodiments the NIR spectral range is used for blood vessel imaging. A spectral window exists from approximately 700 nm to approximately 900 nm, where light can penetrate deep into tissues, and also more radiation is absorbed by venous blood vessels than by surrounding tissues.

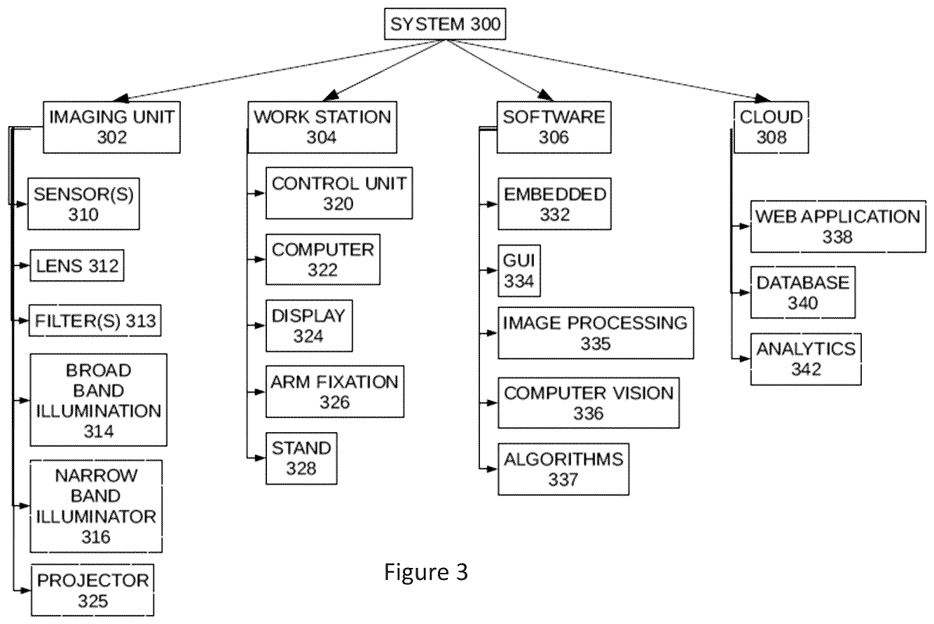

Reference is now made to FIG. 3, which is a simplified block diagram of a system for measuring blood vessels according to an example embodiment of the invention.

FIG. 3 shows a top-level block diagram of an example embodiment system 300. The system 300 may include an imaging/detection unit 302 and a software/computation unit 306.

The imaging/detection unit 302 optionally includes one or more sensor(s) 310, one or more lenses 312, one or more filter(s) 313, one or more illuminator(s) 314 316 and one or more optional projector(s) 325.

In some embodiments the sensor(s) 310 may be CMOS sensor(s).

In some embodiments the sensor(s) 310 may be a multispectral and/or hyperspectral camera(s).

In some embodiments the sensor(s) 310 may be NIR sensor(s) or camera(s).

In some embodiments the lens 312 may optionally be a fixed focal length lens.

In some embodiments the lens 312 may optionally be a zoom lens.

In some embodiments the filter(s) 313 may optionally include bandpass or long-pass filter(s).

In some embodiments the illuminator(s) 314 316 may optionally include NIR LEDS, optionally in a spectral range of 700-1200 nm.

In some embodiments the illuminator(s) 314 316 may optionally include broad band NIR LEDs.

In some embodiments the illuminator(s) 314 316 may optionally include one or more laser sources, optionally in Near IR spectral range of 850 nm and 910 nm.

In some embodiments the illuminator(s) 314 316 may optionally include narrow band illumination, optionally in a spectral range of 900 nm

In some embodiments the illuminator(s) 314 316 may optionally include an array of illuminators.

In some embodiments the optional projector(s) 325 are configured to project colored dots, lines, shapes, text, and such color markings onto a patient's arm.

In some embodiments the software/computation unit 306 optionally includes one or more of a GUI 334, an image processing unit 335, a computer vision unit 336, and a machine learning algorithm unit 337.

In some embodiments the algorithm unit 337 optionally includes one or more of: image processing algorithm(s), vein segmentation algorithm(s), collateral vein detection and/or segmentation algorithm(s), pulse wave detection algorithm(s), and classifier algorithm(s)—optionally machine learning algorithms.

The system 300 may include additional units, such as a work station 304, optional cloud infrastructure 308, etc.

In some embodiments the cloud infrastructure 308 optionally includes one or more of a web application 338, database(s) 340 (optionally including big data analytic capability), and analytic unit(s) 342.

In some embodiments, the work station 304 optionally includes one or more of:
a control unit 320, for controlling operation of the imaging/detection unit 302 and/or one or more of the components of the imaging/detection unit 302;
a computer 322;
a display 324;
an optional organ fixation surface or device 326, for optionally placing an organ at a specific location relative to the illumination 314 316 and/or the sensors 310; and
a stand 328, for placing components of the system at a specific location relative to a patient's organ.

When a medical caretaker (e.g., Family member, nurse or physician) examines a patient's blood vessels, they typically use a three-step procedure: look, listen and feel.

In some embodiments, systems as described herein perform a look, listen and feel based on illuminating and imaging a patient's limb and analyzing the data collected from the imaging.

In some embodiments, methods as described herein performs a look, listen and feel based on illuminating and imaging a patient's limb and analyzing the data collected from the imaging.

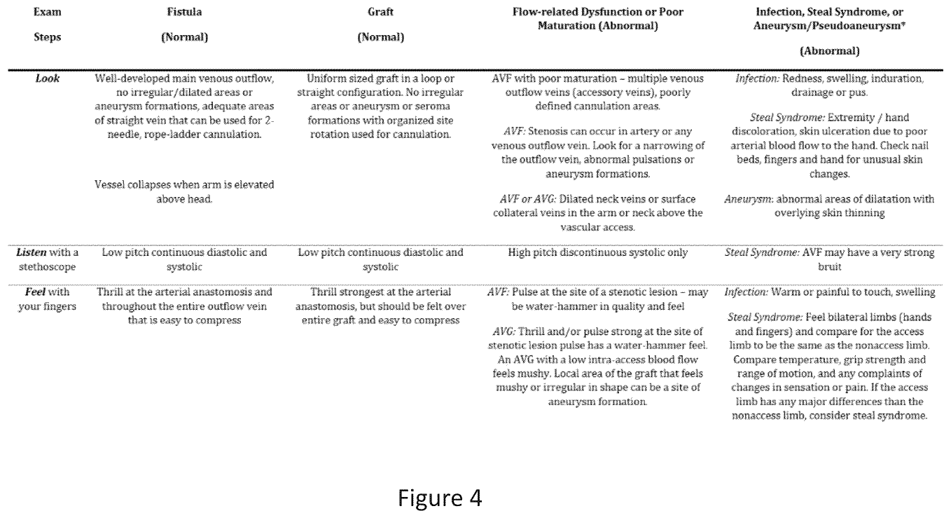

Reference is now made to FIG. 4, which is a table showing a procedure for a medical person to examine a patient with reference to vascular stenotic lesions or thrombosis.

FIG. 4 is intended to show what a human is instructed to do. However, it is known that differences between humans is expected to affect such examinations.

It is noted that automatic examination is potentially able to provide better reproducibility for such examinations.

It is noted that automatic examination is potentially able to provide faster examinations with less involvement of medical staff.

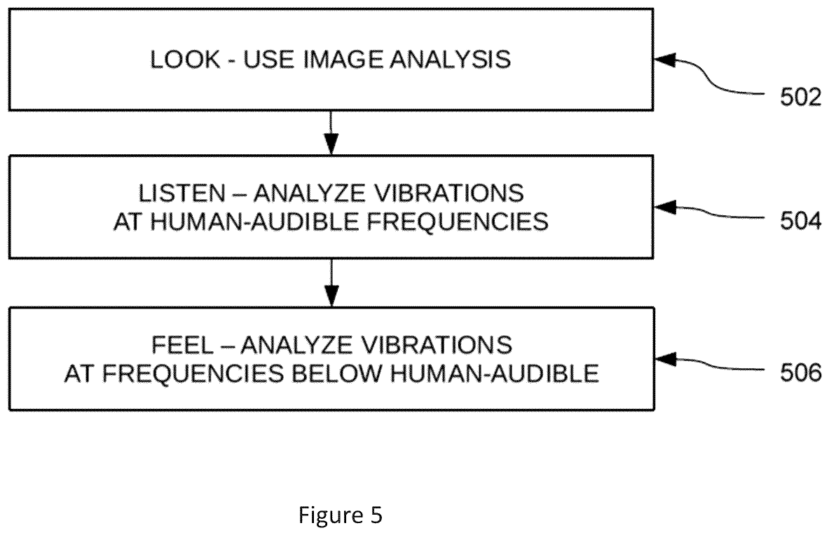

Reference is now made to FIG. 5, which is a simplified flow chart illustration of a method of examining a patient according to an example embodiment of the invention.

The method of FIG. 5 includes:
a device looking (502) at a patient's body by capturing one or more images of the body, and using image analysis on the image(s);
the device listening (504) to a patient's body by capturing vibrations of the body, and analyzing the vibrations at human-audible frequencies; and
the device feeling (506) the patient's body by analyzing vibrations of the body, at frequencies below human-audible frequencies.

In some embodiments, the capturing one or more images of the body is optionally performed by capturing images at Near IR wavelengths.

In some embodiments, the capturing vibrations of the body is optionally performed by laser speckle imaging, as described elsewhere herein.

In some embodiments, the capturing vibrations of the body is optionally performed by a microphone touching the patient's body, and/or by a microphone attached to a stethoscope touching the patient's body.

In some embodiments the capturing vibrations of the body is optionally performed by analyzing a “smearing” or enlarging of a size of one or more spots of light on the body relative to a size of other spots of light on the body.

It is noted that automatic examination, in some embodiments, is potentially able to provide such an examination without a human touching the patient, potentially usable in conditions where medical distancing is desired, such as, for example, when the patient may carry a contagious disease.

In some embodiments, the systems and methods described herein optionally “look”, that is, analyze images of blood vessels, “listen”, that is, analyze vibration of the patient's body at human hearing frequencies, and “feel”, that is, analyze vibration of the patient's body at low frequencies, reaching lower than typical audio frequencies.

In some embodiments, a no-contact surveillance tool is provided, to complement and/or replace physical examination of vascular access (VA). Such surveillance potentially enables early detection of stenosis, potentially earlier than human examination.

In some embodiments, the surveillance tool does not contact a patient's fistula, and/or a patient's limb, even while the limb is optionally positioned in a device which enables position the fistula in a field of view of the device.

In some embodiments, recording and monitoring parameters measure by the surveillance potentially enables the early detection and/or prediction of stenosis, potentially earlier than human examination.

In some embodiments, surveillance is enabled without human touch, for example at distances greater than 10, 20, 30, 40, 50 centimeters from a location of VA.

In some embodiments, the system and methods optionally enable acquiring all parameters typically acquire by a human physical examination by look, feel, and listen.

In some embodiments, it is easier to train persons to operate monitoring VA using embodiments as described herein than using the human senses.

Using embodiments as described herein, potentially add value by recording and using same-patient historical data and tracking changes.

Using embodiments as described herein potentially enable pre- and/or post-session examination in a clinic with no physical contact.

Using embodiments as described herein potentially support medical care under COVID-19. Using embodiments as described herein potentially enable care in a home setup, possibly operated by a patient.

In some embodiments, the projector (e.g., projector 225 and/or 325 of FIGS. 2 and 3 respectively, or other projectors described herein) optionally projects light onto the location of interest to enable a user to locate the patient's body correctly.

In some embodiments, the location of interest is a patient's fistula.

In some embodiments, more than one spot is illuminated simultaneously.

In some embodiments, one location of interest where a spot is illuminated is a patient's fistula, and another location of interest where a spot is illuminated is a location neighboring the patient's fistula, but not at the fistula.

In some embodiments, one location of interest where a spot is illuminated is a fistula aneurism, and another location of interest where a spot is illuminated is a location neighboring the fistula aneurism, but not at the fistula aneurism.

In some embodiments, the projector is a Digital Light Processing (DLP) projector.

In some embodiments, the projector is a laser projector.

In some embodiments, a location of interest, for example a fistula, or an aneurysm, or a bloated area of a body, is optionally identified by using structured lighting and image analysis, and the projector is controlled, optionally automatically controlled, to illuminate the location of interest.

In some embodiments, the DLP and/or the laser projector are optionally controlled to illuminate the location of interest.

In some embodiments, a physician or nurse controls the illumination to the location of interest.

In some embodiments, a physician or nurse controls laser illumination to the location of interest.

In some embodiments, the projector is optionally capable of projecting light in multiple modes. The modes include two or more of:
projecting uniform (or approximately uniform) lighting on an area, or a limited spot, on a patient's body, potentially sufficient for imaging collateral veins;
projecting structured lighting, optionally including stripes of specific widths, equal widths or unequal widths as programmed or other patterns; and
projecting one or more area(s) of coherent laser light, potentially useful for measuring one or more of vibration, micro vibration, and pulses, for example by Laser Speckle Interferometry.

In some embodiments the projector is capable of switching between any one of three different lighting modes: uniform, various line pattern(s) and various spot patterns.

In some embodiments the projector is capable of providing one or more spots, each spot sized in a range of diameters between 0.2 mm and 0.5, 1, 2, 6, 9, 12, 15 or 18 mm on a patient's limb. For example, a spot size of approximately 1 mm. In some embodiments, multiple spots are provided, on an area on the patient's arm. In some embodiments the area is optionally determined by a user of the system, to project onto an area of interest such as an AV fistula. In some embodiment, the area optionally extends entirely across a patient's arm. In some embodiments the area extends entirely across a patient's arm and along the patient's arm for a length in a range between 3 cm and 30 cm.

In some embodiments the projector includes one or more LEDs and/or laser light sources, optionally at Near IR wavelengths.

In some embodiments the projector is optionally a Digital Light Processing (DLP) projector.

In some embodiments the projector optionally includes nano-mirrors to shape light.

In some embodiments the projector optionally includes Micro-Electro-Mechanical System (MEMS) mirrors to shape light.

In some embodiments the projector optionally includes a Digital Mirror Driver (DMD).

In some embodiments, the projector and the camera are packaged in one package.

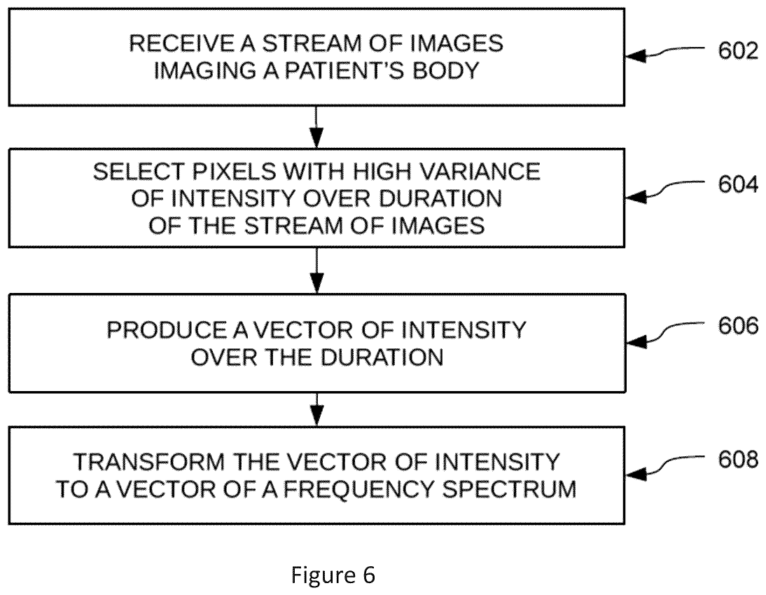

Reference is now made to FIG. 6, which is a simplified flow chart illustration of a method for transforming data from a stream of images to a frequency spectrum according to an example embodiment of the invention.

The method of FIG. 6 includes:
receiving a stream of images imaging a patient's body (602);
optionally selecting one or more pixels with high variance of intensity over duration of the stream of images (604);
producing a vector of intensity over the duration (606);
transforming the vector of intensity to a vector of a frequency spectrum (608).

In some embodiments, the transforming is performed by a Fast Fourier Transform.

In some embodiments, before analyzing the power spectrum, the power spectrum is optionally normalized. By way of some non-limiting examples, a normalization factor is optionally calculated from: total spectrum energy, peak value, peak to baseline ratio, energy in a specific band width, and so on.

In some embodiments, a reference spectrum measured at a remote location (far from the fistula, on the other hand for example), is used as a reference. Both spectrums may or may not be normalized and the measured spectrum replaces by a difference between the spectra at the different locations.

In some embodiments, skewness or kurtosis of the measured power spectrum or the difference power spectrum are optionally used for estimating flow.

In some embodiments, a measured power spectrum is first fitted to a model, in some embodiments assuming one or more hidden model mixtures, by way of a non-limiting example a Poisson-Gaussian mixture, and model parameters are used as correlators to flow.

In some embodiments, energy in a specific frequency range is used for estimating flow.

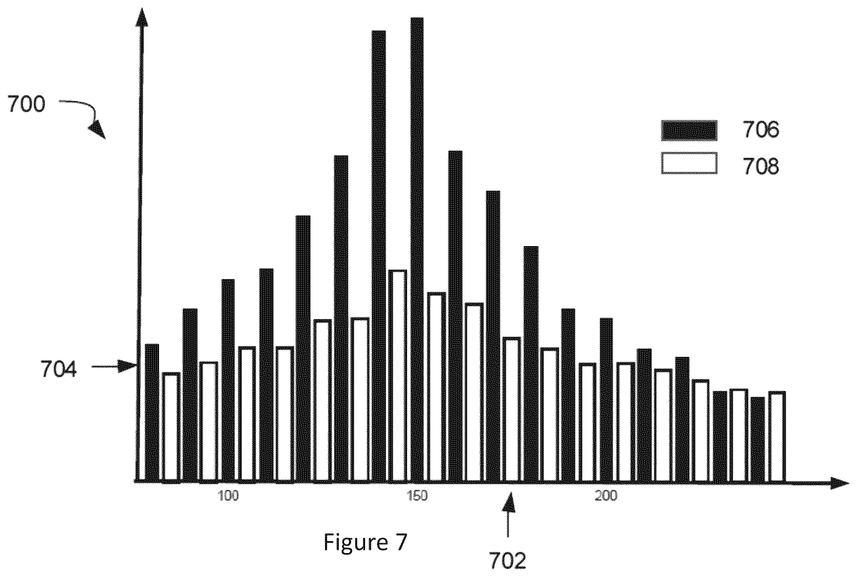

Reference is now made to FIG. 7, which is a graph showing power spectrum of vibrations measured by analysis of images produced by laser speckle imaging.

FIG. 7 shows a graph 700, with an X-axis 702 showing frequency ranges or bins, and a Y-axis 704 showing relative power spectrum in the units in which it was measured.

Two groups of patients were sampled for producing this graph. A first group 706 having a blood flow velocity (FV) greater than 500 mL/minute, and a second group 708 having FV less than 500 mL/minute.

The graph 700 shows us that the maximum in the power spectrum is located approximately at approximately 140 Hz for both groups. This leads us to suspect that listening to the pitch of the blood flow in both groups might not be a good method to differentiate among them. However, analyzing the power spectrum of both groups shows differences:

The first group 706 appears to have a higher amplitude at the maximum than the second group 708;

The second group 708 appears to have a flatter, or broader, curve than the first group 706.

The vibrations analyzed in the power spectrum, as shown in FIG. 7, are apparently caused by blood flow and/or turbulence through a blood vessel.

It should be understood that the graph shown in FIG. 7 is an example provided to allow a person having skills in the art to understand the invention. For example, while the frequencies shown in FIG. 7 reach about 250 Hz, the present invention should not be limited to that number. In some embodiments, the system is configured to generate up to and optionally above 1000 Hz.

Flow and turbulence change over time and are affected by local physical conditions in and around the vessels through which the flow occurs. The physical conditions potentially include a pressure gradient, vessel diameter, vessel wall compliance, vessel inner surface characteristics, and so on.

The power spectrum of blood flow measured at VA/fistula locations is potentially related to physical and/or clinical flow conditions at these locations. Changes in the features of such power spectra over time potentially correlate to degradation in fistula health. Analyzing the changes in the power spectrum obtained from the VA/fistula location are potentially early stage predictive of fistula deterioration.

It is noted with reference to early-stage predictions described herein, that such predictions potentially enable performing percutaneous transluminal angioplasty (PTA) earlier than would be performed based on the existing state of medical examination.

In some embodiments, the power spectrum is measured by measuring an intensity of light reflected off a patient's body. The intensity is expected to change at a frequency related to frequency of vibration of the body.

In some embodiments, the power spectrum is measured by measuring an intensity of light reflected off an illumination area on the patient's body. In such embodiments the vibration is practically measured specifically at the illuminated area.

In some embodiments, the power spectrum is measured by measuring differences between successive images of the body, for example small shifts of a pattern on the body. The pattern may be a mole on the skin, structured lighting, movement of a spot of light, movement of more than one spot of light, movement of laser speckles, and similar movements.

### Performing Look, Listen and Feel

We additionally describe some aspects of the invention in terms of a procedure of “Look, Listen and Feel” which is used by medical staff.

In some embodiments, a “Look, Listen and Feel” procedure is optionally performed by embodiments of the system described herein.

In some embodiments, systems as described herein perform a look, listen and feel based on illuminating and imaging a patient's limb and analyzing the data collected from the imaging.

In some embodiments, methods as described herein performs a look, listen and feel based on illuminating and imaging a patient's limb and analyzing the data collected from the imaging.

A fistula bruit, also called a vascular murmur, is an indicator of how dialysis access is functioning.

An arteriovenous fistula is one access type that is created by connecting an artery to a vein under the skin, usually in the upper or lower arm or leg. (i) The high blood flow from the artery through the vein allows the fistula to grow larger and stronger. A healthy AV fistula has a bruit (a rumbling sound that a human can hear), a thrill (a rumbling sensation that a human can feel), and good blood flow rate.

In some embodiments, the “Look” aspect is optionally performed by imaging a body or limb and analyzing an image or images to quantify blood vessel structure and/or fistula structure.

In some embodiments, the “Look” aspect is optionally performed by imaging a body or limb using structured light, and producing a 3D shape of a fistula.

In some embodiments, the “Listen” aspect is optionally performed by measuring vibrations of a body or limb and analyzing the vibrations to quantify parameter values relating to a medical condition of a fistula. In some embodiments, the “Listen” aspect includes analyzing vibrations in a frequency range within the human audible range.

In some embodiments, the “Feel” aspect is optionally performed by measuring vibrations of a body or limb and analyzing the vibrations to quantify parameter values relating to a medical condition of a fistula. In some embodiments, the “Feel” aspect includes analyzing vibrations optionally in a frequency range extending even beyond and/or below the human audible range.

In some embodiments, analyzing vibrations is optionally performed in a frequency range of less than 1,000 Hz. In some embodiments, analyzing vibrations is optionally performed in a frequency range of less than a typical human speech, for example less than 4,000 Hz.

By way of some non-limiting examples, the “Feel” aspect includes one or more of:

Measuring human pulse, which is typically in a range of 40 beats per minute and above. Such measurement needs analyzing vibrations at a frequency of 1 Hertz and even somewhat less. When such analyzing is performed by analyzing image frames of a video sequence, it is sufficient to analyze image frames at approximately double the rate of the frequency being measured, that is, for example, approximately 2 frames per second or above.

Measuring thrill, which is typically in a range of 50-250 Hertz or 50-750 Hertz. When such analyzing is performed by analyzing image frames of a video sequence, it is typically sufficient to analyze image frames at approximately double the rate of the frequency being measured, that is, for example, approximately 100 frames per second or above.

Analyzing and quantifying a power spectrum of the vibrations, for example as described above with reference to FIGS. 6 and 7.

In some embodiments, the “Look, Listen and Feel” is performed without physically touching the patient, by image analysis and/or by using a specific mode of lighting.

Reference is now made to FIG. 8, which is a simplified illustration of a system for measuring blood vessels according to an example embodiment of the invention.

FIG. 8 depicts a system 800 which includes at least one illumination source 804 and/or projection component 804 and at least one detector 802, such as a camera.

FIG. 8 also depicts some optional functions performed by the system 800.

A first optional function 810 is illumination by Near IR, which potentially enables imaging of subdermal organs such as veins and/or AV fistulas.

A second optional function 820 is illumination by structured light, which potentially enables determining a 3D structure a surface of a patient's hand 822.

A third optional function 830 is an optional projection of colored dots, lines, shapes, text, and such color markings 834 onto a patient's arm 832. In some embodiments such a projection serves as a graphical user interface, projected onto the patient arm. In some embodiments the optional markings 834 include data such as numbers and/or text projected onto the patient's arm, for example the results of readings. In some embodiments the optional markings 834 include suggested treatment instruction, optionally based on analysis of images collected by the detector 802. In some embodiments the optional markings 834 include marking a location of an AV fistula. In some embodiments the optional markings 834 include marking an area on the arm which is detected by analysis of movement of the skin to vibrate, for example in an optical image analysis corresponding to vibration analysis, which itself corresponds to a “look” or a “listen” or a “feel” action performed by a caregiver.

A fourth optional function 840 is an optional analysis of vibration using laser speckle illumination. The laser speckles shift as a result of vibration of the patient's skin caused by blood flow, at various frequencies. Some of the frequencies correspond to a human performing a “listen” action, that is, the frequencies are at human-audible frequencies. Some of the frequencies correspond to a human performing a “feel” action, that is, the frequencies are below typical human-audible frequencies. It is noted that a vibration map, optionally at locations correctly registered to the patient's body, may be projected onto the patient's body, displaying where the patient's skin is detected to vibrate. In some embodiments, the vibration map shows where vibrations of the patient's skin exceed a threshold value. In some embodiments, the vibration map shows where vibrations of the patient's skin are below a threshold value. The threshold value is optionally set to be in a range between zero (showing all locations where vibration is detected) and up to 90% or even 99% of a maximum vibration value. In some embodiments a contactless, machine vision-based, monitoring system is provided, such as shown, by way of a non-limiting example, in FIGS. 1, 2, 3, and 8.

In some embodiments the system includes one or more illumination source(s) 804 and/or projection component 804, optionally using Digital Light Processor (DLP) architecture.

In some embodiments the detector(s) 802 is optionally a camera, by way of a non-limiting example a fast CMOS sensor arranged to capture imaging, optionally using multi-modal imaging such as 3D imaging (for example using structured lighting), 2D imaging, Near-IR imaging, visible light imaging, laser speckle imaging.

In some embodiments the light source(s) include wavelengths in Near IR and in visible wavelengths.

In some embodiments detector(s) or camera(s) 802 and the illumination source(s) 804 observe and illuminate a same area.

Depending on a distance of the area from the system 800, an angle between the detector(s) or camera(s) 802 and the illumination source(s) 804 may be adjusted. In some embodiments one or more of the detector(s) or camera(s) 802 and the illumination source(s) 804 may be mounted to be adjustable, optionally automatically adjustable, so observe and illuminate the same area.

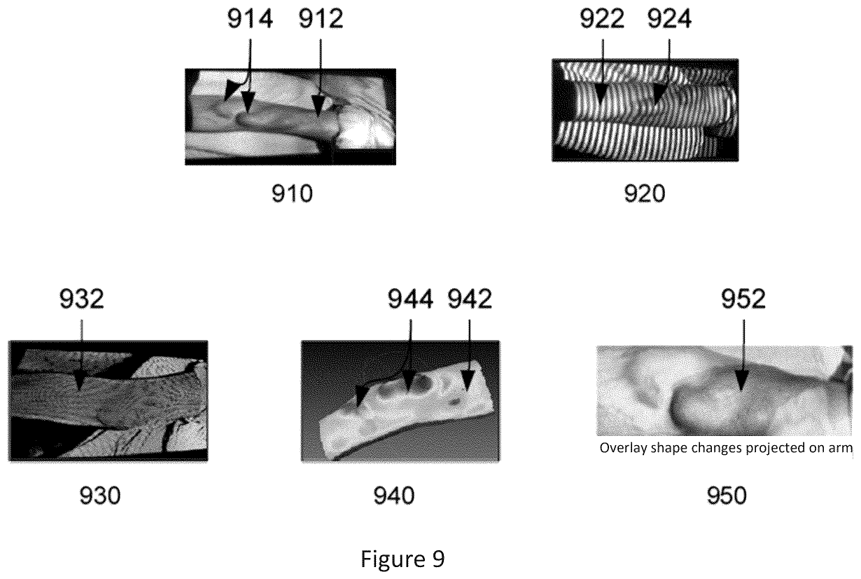

Reference is now made to FIG. 9, which is a simplified illustration of optional lighting modes and optional images captured according to an example embodiment of the invention.

FIG. 9 shows:
a first image 910 in which a patient's arm 912 is illuminated in Near-IR lighting, which enables image capture and analysis which potentially enable vein detection and measurements, optionally including collateral veins. The first image 910 also shows fistulas 914;
a second image 920 in which the patient's arm 922 is illuminated by structured light, which enables image capture and analysis which potentially enable 3D analysis of the arm shape and potentially enables detection of vibration and/or pulsation of the arm or the fistulas. The second image 920 also shows fistulas 924;
a third image 930 which shows 3D shape of the patient's arm 932, the display of 3D shape being, by way of a non-limiting example as a point cloud image and/or as equal elevation contour lines;
a fourth image 940 which shows 3D contour analysis of the patient's arm 942, showing, for example an elevated portion 944 of the patient's arm 942; and
a fifth image 950 which shows a projection 952 of shape changes projected onto the patient's arm;

Monitoring a shape of a patient's fistula potentially enables detecting aneurysm(s) and/or edema(s).

In some embodiments the image analysis and shape change detection are performed on at least two images taken at different stages during an arm elevation test. In some embodiments the registration of arm shape features potentially enable comparing two images captured at different arm elevations.

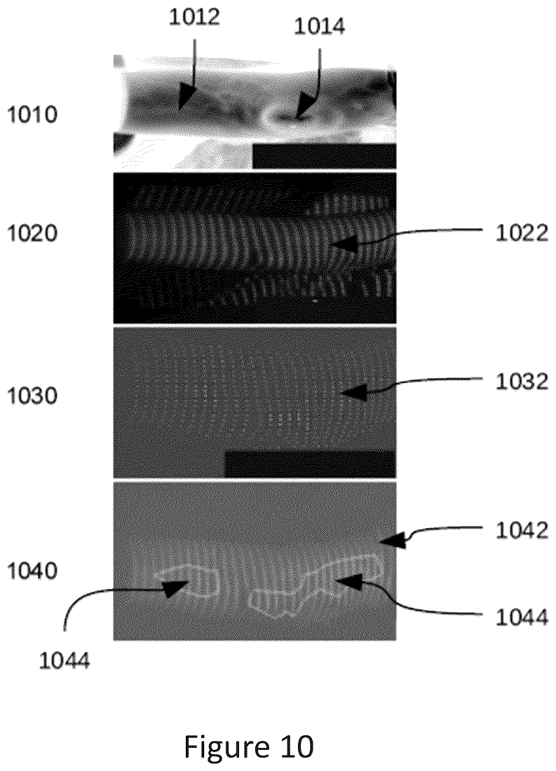

Reference is now made to FIG. 10, which is a simplified illustration of laser vibrometry employed to “listen” to a fistula according to an example embodiment of the invention.

FIG. 10 shows:
a first image 1010 in which vibrometry is used on a patient's arm 1012. An area 1014 on the patient's arm 1012 is illuminated by coherent light, which potentially enables accurate vibration measurement of a specific area of the patient's arm, and/or accurate detection of changes in blood flow in the specific area of the patient's arm;
a second image 1020 in which a pattern is projected onto the patient's arm 1022, which potentially enables area vibrometry, mapping vibrations and/or pulsatility and or results of measurements obtained by analyzing one or more images over an area of the patient's arm. Such area monitoring potentially enables detecting potential locations for inserting needles into the patient's arm;
a third image 1030 which shows an optional pulsatility or vibration mapping 1032 of the patient's arm. In some embodiments the mapping includes projecting spots at different intensities and/or different colors, based on a value calculated for the pulsatility and/or vibration at a location of the spot; and
a fourth image 1040 which shows an optional projection of the pulsatility or vibration mapping 1044 onto the patient's arm, optionally marking areas where the pulsatility or vibration are greater than a threshold level.

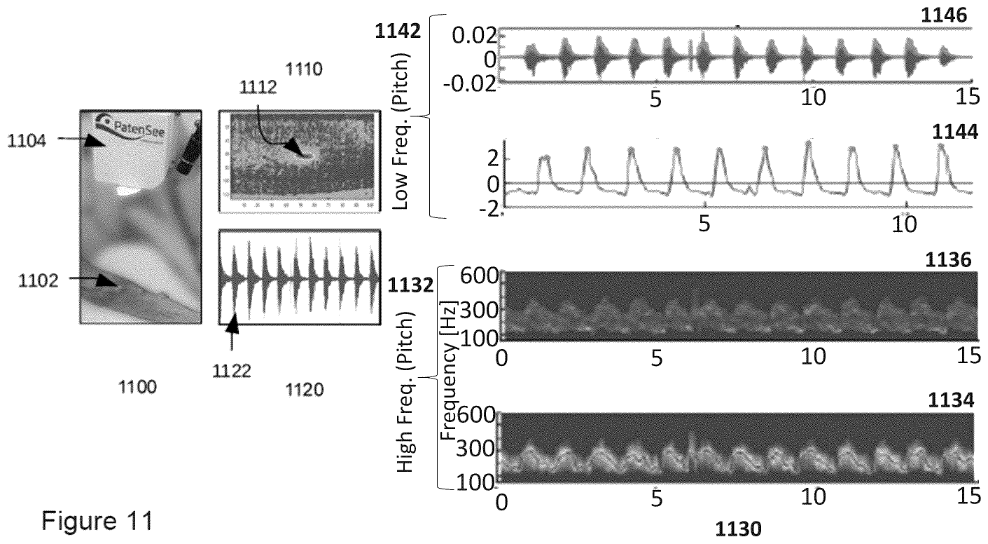

Reference is now made to FIG. 11, which is a simplified illustration of laser speckle vibrometry employed to assess vibrations according to an example embodiment of the invention.

FIG. 11 shows:
a first image 1100 which shows a system 1104, such as the system 100 shown in FIG. 1 and/or the system 800 shown in FIG. 8, used to assess a patient's arm, by projecting light onto an area 1102 on the patient's arm;
a second image 1110 which shows a speckle acquisition image of the area 1112;
a third image 1120 which shows microvibration 1122 of the area 1112 of the second image 1110;
a fourth image 1130 which shows spectral analysis graphs 1134 1136 1144 1146. The fourth image 1130 shows two spectral analysis graphs 1134 1136 in a higher frequency domain which corresponds to spectral analysis in a pitch domain, typically at frequencies at or above 50 Hz or even 100 Hz, which potentially enables blood flow analysis and two spectral analysis graphs 1144 1146 in a lower frequency domain which corresponds to spectral analysis in a pulse domain, typically at frequencies at or less than 100 Hz or even 50 Hz, which potentially enables analysis of the patient's pulse. In some embodiments, the above-mentioned pitch domain corresponds to frequencies in a bruit range. In some embodiments, the above-mentioned pulse domain corresponds to frequencies in a thrill range.

Laser speckle vibrometry potentially enables assessing one or more of: micro-vibrations; pitch (flow noises and/or bruit); pulsatility, pulse intensity and/or thrill.

In some embodiments, features extracted from one or more of the above-mentioned imaging modalities are optionally used to train machine learning algorithms to assess a potential of stenosis.

In some embodiments visible light sources built into the hybrid DLP platform are optionally used to project layers of information and/or GUI elements onto the patient arm to support caregivers and/or the patient.

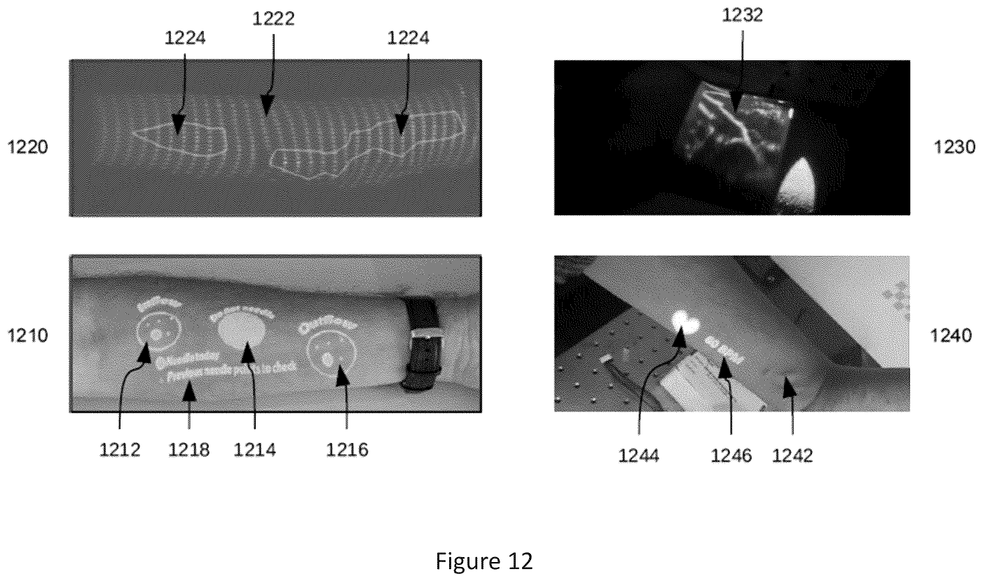

Reference is now made to FIG. 12, which is a simplified illustration of various projections onto a patient's arm according to an example embodiment of the invention.

FIG. 12 shows:
a first image 1210 in which a sample GUI projection onto the patient's arm is shown. The first image 1210 shows a first candidate area 1212 for using a needle for inflow, a second candidate area 1216 for using a needle for outflow, a third area 1214 marked as “do not needle” and a fourth area where suggested treatment instructions are projected;
a second image 1220 in which multiple light spots are projected onto the patient's arm 1222, and areas 1224 are marked where pulsatility or vibration or other calculated values as described herein are analyzed to be greater than a specified threshold. In some embodiments the light spots are optionally projected at an intensity and/or a color corresponding to a value of the pulsatility/vibration/calculated value;
a third image 1230 in which a vessel mapping is projected onto the patient's arm. The vessel mapping potentially assists needle insertion into a vascular access location. The mapping may include annotations; and
a fourth image 1240 in which real-time physiological monitoring data is projected onto the patient's arm.

It is noted that the areas 1224 may be good candidate(s) for needling, potentially being areas selected for a needling plan. The areas 1224 may also indicate where a fistula is located.

In some embodiments one or more of the first candidate area 1212 for using a needle for inflow, the second candidate area 1216 for using a needle for outflow, and the third area 1214 marked as “do not needle” are optionally determined automatically based on one or more of:
distance of one candidate area to another candidate area;
distance from locations where a fistula is located in the patient's arm, based on historic or previous data;
a plan allocating candidate locations for future needle insertions; and
optionally, a plan for re-using previous fistula locations.

It is noted that candidate areas marked for needling, such as the first candidate area 1212 and the second candidate area 1216 of image 1201, may be marked by illuminating the candidate areas, by illuminating a line outlining the areas, by illuminating the area surrounding the candidate areas, by a symbol such as an “X” at a location or vicinity where a needle is suggested to be inserted, and other means for indicating an area by illumination.

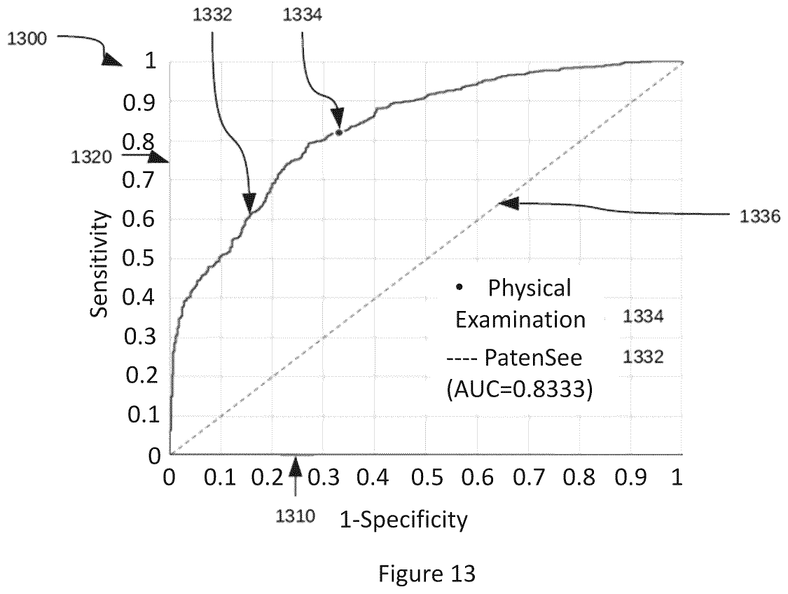

Reference is now made to FIG. 13, which is a graph which shows a Receiver Operator Characteristics (ROC) curve of a stenosis detecting model according to an example embodiment.

FIG. 13 shows a graph 1300 having an X-axis 1310 showing specificity and a Y-axis 1320 showing sensitivity, an ROC curve 1332, a specific point 1334 and a line 1336 which corresponds to a 50/50 chance of any model obtaining a correct result

The specific point 1334 of FIG. 13 is located at a Specificity-Sensitivity location corresponding to a best-possible physical examination.

FIG. 13 shows an ROC curve 1332 of a stenosis detecting model according to an example embodiment compared to stenosis as detected by Doppler sonography.

The stenosis detecting model was trained on 1139 observations (from 44 patients) with 5-fold cross validation.

It is noted that the Area Under the Curve (AUC)=0.83.

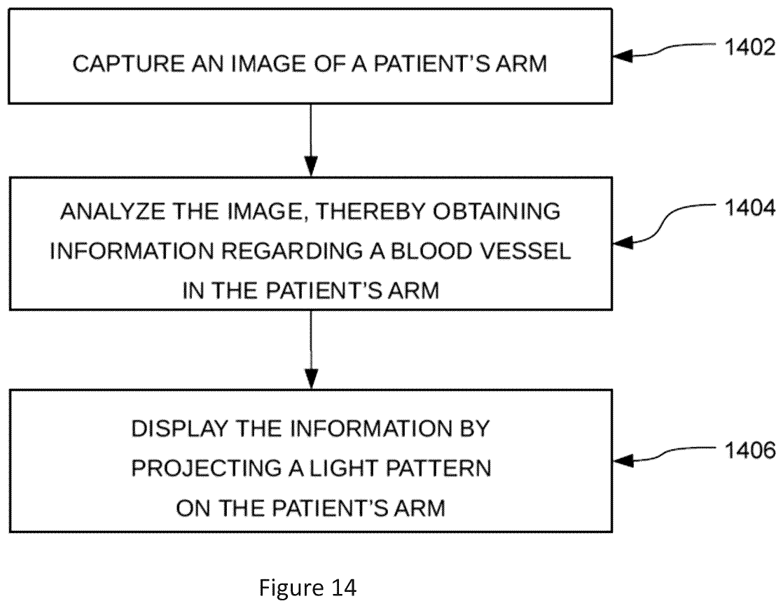

Reference is now made to FIG. 14, which is a simplified flow chart illustration of a method for displaying data on a patient's arm according to an example embodiment of the invention.

The method of FIG. 14 includes:
capturing an image of a patient's arm (1402);
analyzing the image, thereby obtaining information regarding a blood vessel in the patient's arm (1404);
displaying the information by projecting a light pattern on the patient's arm (1406).

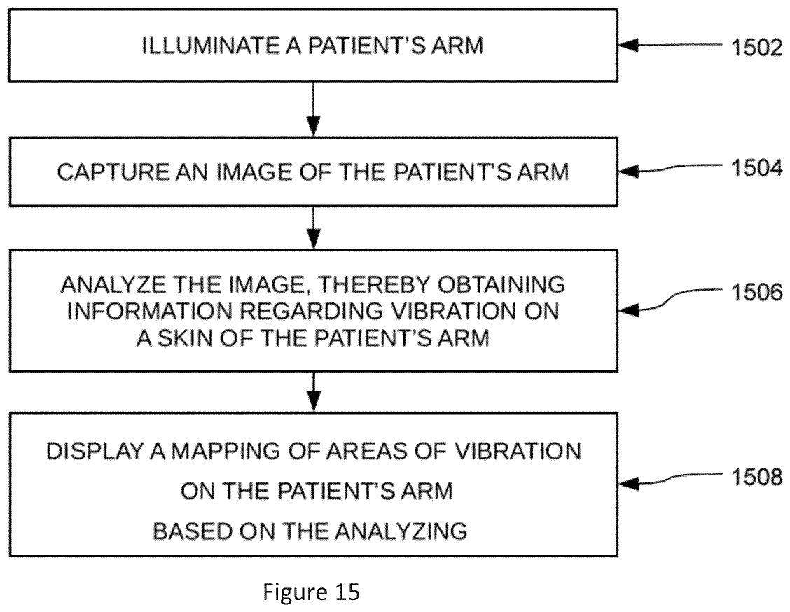

Reference is now made to FIG. 15, which is a simplified flow chart illustration of a method for mapping areas of vibration on a patient's arm according to an example embodiment of the invention.

The method of FIG. 15 includes:
illuminating a patient's arm (1502);
capturing an image of the patient's arm (1504);
analyzing the image, thereby obtaining information regarding vibration on a skin of the patient's arm (1506); and
displaying a mapping of areas of vibration on the patient's arm based on the analyzing (1508).

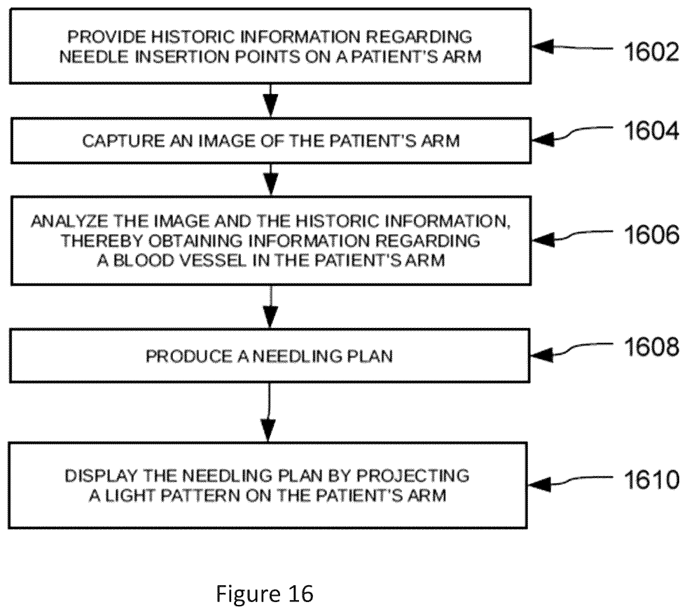

Reference is now made to FIG. 16, which is a simplified flow chart illustration of a method for displaying a needling plan on a patient's arm according to an example embodiment of the invention.

The method of FIG. 16 includes:
providing historic information regarding needle insertion points on a patient's arm (1602);
capturing an image of the patient's arm (1604);
analyzing the image and the historic information, thereby obtaining information regarding a blood vessel in the patient's arm (1606);
producing a needling plan (1608); and
displaying the needling plan by projecting a light pattern on the patient's arm (1610).

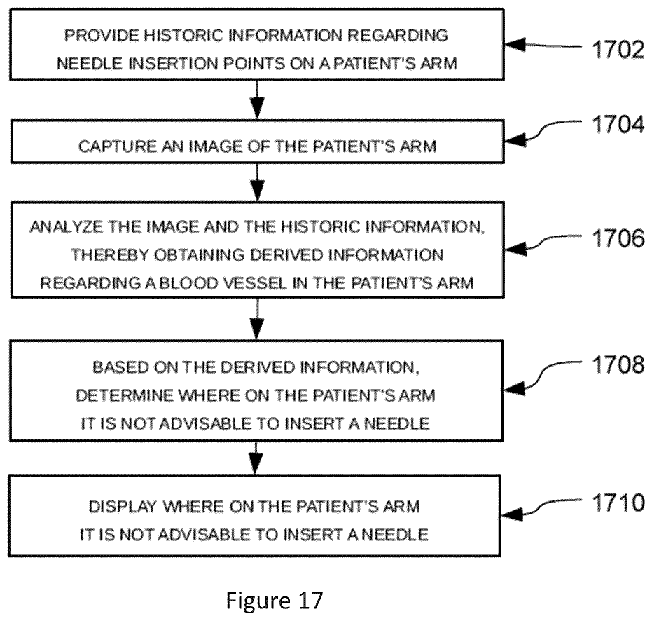

Reference is now made to FIG. 17, which is a simplified flow chart illustration of a method for displaying where not to insert a needle on a patient's arm according to an example embodiment of the invention.

The method of FIG. 17 includes:
providing historic information regarding needle insertion points on a patient's arm (1702);
capturing an image of the patient's arm (1704);
analyzing the image and the historic information, thereby obtaining derived information regarding a blood vessel in the patient's arm (1706);
based on the derived information, determining where on the patient's arm it is not advisable to insert a needle (1708); and
displaying where on the patient's arm it is not advisable to insert a needle (1710).

In some embodiments the displaying where on the patient's arm it is not advisable to insert a needle is by projecting a light pattern on the patient's arm.

Various embodiments and aspects of the present invention as delineated hereinabove and as claimed in the claims section below find experimental and calculated support in the following examples.

### Examples

### Exemplary General Information Regarding Acquisition.

In some embodiments, optionally, there is a process of calibrating the system, which is done periodically (once a day, or once a month, for example), without the presence of patients.

In some embodiments, generally speaking, there are two acquisition processes-vibration measurement and 3D generation process. In some embodiments, the vibration measurement is performed once in each session, and the three-dimensional measurement is performed optionally twice-once when the hand is placed parallel to the floor, and a second time when the hand is lifted at an angle above the heart (also referred to as “elevation test”). In some embodiments, the lifting is performed above the heart, at a height which will cause the fistula to empty (or not empty).

In some embodiments, the acquisition processes can be divided into sub-tasks, which are completed in order to generate the raw information.

The following table summarizes an exemplary process divided by sub-tasks.

| Acquisition | | |
|---|---|---|
| Process | Subtask | Description Subtask |
| Vibration | Return to a | The medical caretaker (e.g., nurse) or a mechanical tool sees where |
| | previous | the measurement was made the last time, and the system will be |
| | measurement | placed on the patient's hand according to the location of the |
| | point | previous measurement. Once it is positioned, the camera will take |
| | | several photos to allow the next session to focus on the same spot |
| | | again. |
| | Finding ROI | Finding the area on the CCD surface where the laser beam falls |
| | | (single spot). |
| | Vibration | Recording high-speed vibrations on the spot placed during the sub- |
| | photography | task “Return to a previous measurement point” + additional spots |
| | | along the ROI |
| 3D modeling | Calibration | Performed periodically without the presence of patients. The |
| | | system takes several images of known scenes (for example, a |
| | | chessboard), and extracts parameters of the camera and the |
| | | projector in relation to the world. |
| | Structured | The camera takes a sequence of images in sync with the projector. |
| | light | One of the images is full illumination, and the other images are |
| | | images of horizontal and vertical stripes, which allow a three- |
| | | dimensional reconstruction of the object. |

In some embodiments, specific requirements are used in each sub-task. For example, in relation to a sub-task of photographing the vibrations (as described herein elsewhere) the lens are configured to have a large focal length.

In some embodiments, another hardware-related feature is the CCD's region of interest (ROI). In some embodiments, a full ROI may limit the frame rate, whereas a limited ROI may allow higher frame rates.

The below are exemplary descriptions of several of the tasks and sub-tasks.

### Purchase Process: Vibrations:

Sub-task—Return to a previous measurement point: a laser marks a central point, while a picture of the fistula with the central point from last time marked on it is displayed on a screen. In some embodiments, the image is with maximum FOV, and contains both the fistula and its surroundings.

The central point is to be directed on the patient's hand so that it lights up in a similar place as previously.

The system will then take several pictures to prepare the picture of the hand and the point previously aimed at, so that in the next session there will be a record of where the measurement was made.

Subtask—Finding ROI: The lens then changes the focal length to maximum. The laser projector continues to be aimed at the same place. The camera takes several pictures, and the software locates, with the help of image processing tools, where in the image's pixel space the laser illumination falls.

Subtask—Vibration Photography: The ROI may be changed from a full ROI to a limited ROI. Reducing the number of pixels allows shooting at a higher rate.

The projector now illuminates a collection of dots, and the camera captures them all at once for several seconds.

Option to repeat the process with slight moves of the points on both axes.

### Purchase Process: 3D Modeling.

Sub-task—calibration: once in a while (for example once a day, or once a month, but not during the session itself) the system is calibrated using a calibration board. For example, a checkerboard style board is used. In some embodiments, the calibration saves internal data (for example, center or focal length) and external data (for example, angle relative to the world) of the camera and the projector.

Subtask—Structured light: will be performed optionally twice in the session, once when the hand is parallel to the floor and a second time when the hand is raised and the fistula is emptied (or partially emptied, or not emptied).

In the session itself, the patient puts his hand under the projector, and the projector takes several pictures of stripes along and across. With the help of triangulation calculations, and with the help of the calibration providers, it is possible to perform depth reconstruction and extract a point cloud of the photographed hand—that is, a collection of XYZ points in space. In some embodiments, collection of these points are used for the generation of a 3D object of the hand with the fistula.

In some embodiments, the whole process is fully automated.

In some embodiments, the procedure is repeated after raising the hand above the heart level and letting the blood in the fistula drain.

In some embodiments, a potential advantage of the system of the invention is that it uses both vibration photography and Structured light photography, while taking advantage of the flexibility of a mechanized lens and controlling the ROI and the shooting rate, all within a one unique system.

In some embodiments, optionally, additional acquisition processes are performed, such as three-dimensional photography, collection of historical information, and its projection on the patient.

### Exemplary Raw Data Acquired During the Acquisition Processes:

In some embodiments, raw data information is obtained directly from the camera, or with the help of processing processes. In some embodiments, raw data information is used for generating processed information that is presented to the medical caretaker (e.g., nurse) and/or that is used to train the ML system.

The following table summarizes exemplary raw data information acquired, for example, during the different sub-tasks.

| | Acquisition | | |
|---|---|---|---|
| # | Process | Subtask | Description of the information |
| 1 | Vibration | Return to a | An image of the patient's hand in as large a FOV as possible and |
| | | previous | a full ROI, and on the image is marked a point where the system |
| | | measurement | focused during the last photograph. |
| | | point | |
| 2 | Vibration | Vibration | Vibration of the central point, which was marked by the system |
| | | photography | or others. The vibrations of each frame in the image are analyzed |
| | | | in relation to the previous frame using the Optical Flow |
| | | | algorithm, and receive a value that represents the displacement |
| | | | of the pixels. For example, the value “2” indicates that in the |
| | | | current frame, in relation to the previous frame, there was an |
| | | | average shift of 2 pixels. |
| 3 | Vibration | Vibration | Vibrations of additional spots. |
| | | photography | |
| 4 | Vibration | Vibration | Improving the quality of the signal in 2 by using vibrations close |
| | | photography | to 2 taken in 3, for example with the help of Adaptive filtering. |
| 5 | Vibration | Vibration | A complete transformation of 4 |
| | | photography | |
| 6 | Vibration | Vibration | High pass filtering of high frequencies of 5, for example |
| | | photography | frequencies above 150 Hz, frequencies above 200 Hz, |
| | | | frequencies above 250 Hz, etc. |
| 7 | Vibration | Vibration | Low pass filtering of low frequencies of 5, for example |
| | | photography | frequencies below 50 Hz. |
| 8 | 3D | Calibration | A description of the camera and the projector in relation to |
| | modeling | | themselves, and a description of them in relation to the world. |
| 9 | 3D | Structured | Because one of the pictures taken in structured light is full |
| | modeling | light | lighting, pictures of the hand and the fistula before and after the |
| | | | arm elevation test are obtained. |
| 10 | 3D | Structured | Using structured light and the calibration matrices to perform a |
| | modeling | light | 3D reconstruction of the object. |

Exemplary Processed Information to be Displayed to the User and/or to the ML:

In some embodiments, the system comprises two modes-a manual monitoring system mode during which the medical caretaker looks at processed information to make decisions about the patient; and an automatic monitoring system mode in which an ML system provides recommendations without the need of external human interaction.

In some embodiments, in either case, raw data information is used in both modes.

### Exemplary Use of Raw Data Information in the Process:

| | | | | |
|---|---|---|---|---|
| | | | Information to be | |
| Source of raw | Description of the | | presented to | |
| data (from table | information | The information | manual | Information to be |
| above) | processed | format | monitoring | used for ML |

LOOK

| | | | | |
|---|---|---|---|---|
| 9 | Directly display | A collection of | Comparison | |
| | the acquired | greyscale images | between images | |
| | images and the | across sessions | throughout the | |
| | raw data. Because | during the | follow-up period | |
| | the images are | tracking period of | in relation to the | |
| | taken in near | a hand at rest | current image. | |
| | infrared, the | parallel to the | Identification of a | |
| | blood vessels | floor. | change for the | |
| | around the fistula | | worse, in a | |
| | are more | | manner similar to | |
| | prominent than in | | a nurse's view of | |
| | a normal RGB | | the fistula. | |
| | image. | | | |
| 9 | Directly display | A collection of | Comparison | |
| | the acquired | greyscale images | between images | |
| | images and the | across sessions | throughout the | |
| | raw data. Because | during the | follow-up period | |
| | the images are | tracking period of | in relation to the | |
| | taken in near | a hand at rest | current image. | |
| | infrared, the | perpendicular to | The nurse's view | |
| | blood vessels | the floor or in a | after raising the | |
| | around the fistula | lifting machine. | hand, and | |
| | are more | | examination of | |
| | prominent than in | | the emptying of | |
| | a normal RGB | | the fistula. | |
| | image. | | | |
| 10 | Fistula volume | Graph. The x-axis | A decreasing | Extracting |
| | ratio before and | is the session | ratio indication | features from the |
| | after the arm | time, the y-axis is | indicates a future | graph, for |
| | elevation test. | the ratio result. | stenosis. | example average |
| | | | | slope and |
| | | | | standard |
| | | | | deviation, which |
| | | | | will be used as |
| | | | | input to a learning |
| | | | | system. |
| 10 | The volume of | Graph. The x-axis | | Extracting |
| | the fistula | is the session | | features from the |
| | | time, the y-axis is | | graph, for |
| | | the volume (for | | example average |
| | | example in | | slope and |
| | | cm\hat{ }3). | | standard |
| | | | | deviation, which |
| | | | | will be used as |
| | | | | input to a learning |
| | | | | system. |
| 10 | The extent of the | Graph. The x-axis | | Extracting |
| | fistula | is the session | | features from the |
| | | time, the y-axis is | | graph, for |
| | | the circumference | | example average |
| | | (For example in | | slope and |
| | | cm). | | standard |
| | | | | deviation, which |
| | | | | will be used as |
| | | | | input to a learning |
| | | | | system. |

LISTEN

| | | | | |
|---|---|---|---|---|
| 4 | Playback + signal | Playback of the | While listening to | Raw data for ML |
| | at the time of the | signal recorded in | the recorded | system audio |
| | central spot | 4 (wav file) + a | signal, the | recognition in |
| | | graph where the | medical caretaker | combination with |
| | | x-axis is time | will see the signal | visual |
| | | during the | recorded in time | information. |
| | | recording, and the | and a line follows | |
| | | y-axis is the | the surface of the | |
| | | vibrations (as | signal throughout | |
| | | described in 2). | its playback. The | |
| | | | playback | |
| | | | experience will | |
| | | | be similar to | |
| | | | placing a | |
| | | | stethoscope at the | |
| | | | point of contact. | |
| | | | The signal in | |
| | | | previous sessions | |
| | | | can be repeated | |
| | | | and listened as | |
| | | | well. | |
| 6 | The maximum | Graph. The x-axis | An indication of | Extracting |
| | index of the raw | is the session | increasing the | features from the |
| | information in 6. | time, the y-axis is | maximum index | graph, for |
| | Tracking the | the maximum | over several | example average |
| | index throughout | index in Hz. | sessions may | slope and |
| | the tracking | | indicate a | standard |
| | period. | | stenosis. | deviation, which |
| | | | | will be used as |
| | | | | input to a learning |
| | | | | system. |
| 6 + 7 | The ratio between | Graph. The x-axis | An indication of | Extracting |
| | the amplitude of | is the session | an increasing | features from the |
| | the maximum at 6 | time, the y-axis is | ratio may indicate | graph, for |
| | and the maximum | the ratio (no | a stenosis. | example average |
| | at 7. | units). | | slope and |
| | | | | standard |
| | | | | deviation, which |
| | | | | will be used as |
| | | | | input to a learning |
| | | | | system. |
| 6 | The FWHM | Graph. The x-axis | An indication of a | Extracting |
| | (width at half | is the session | decrease in value | features from the |
| | amplitude) value | time, the y-axis is | may indicate a | graph, for |
| | around the | the width in Hz. | stenosis. | example average |
| | maximum point | | | slope and |
| | at 6. | | | standard |
| | | | | deviation, which |
| | | | | will be used as |
| | | | | input to a learning |
| | | | | system. |

FEEL

| | | | | |
|---|---|---|---|---|
| 2 + 3 | Extracting the | Graph. The x-axis | A trend of an | Extracting |
| | heart rate from all | is the session | increase or | features from the |
| | the vibrations | time, the y-axis is | decrease in heart | graph, for |
| | based on the | the heart rate in | rate may indicate | example average |
| | identification of | beats per minute. | an anomaly. | slope and |
| | harmonics in the | | | standard |
| | low frequencies | | | deviation, which |
| | and providing an | | | will be used as |
| | index of the | | | input to a learning |
| | quality of the | | | system. |
| | index of the heart | | | |
| | rate measured in | | | |
| | each spot. The | | | |
| | heart rate will be | | | |
| | determined based | | | |
| | on the spots with | | | |
| | the best quality | | | |
| | index. | | | |
| 2 + 3 | Heart rate quality | Graph. The x-axis | A decrease in the | Extracting |
| | index of the | is the session | ratio may indicate | features from the |
| | central point | time, the y-axis is | a stenosis. | graph, for |
| | relative to the | the ratio of the | | example average |
| | best heart rate | quality of the | | slope and |
| | index quality that | heart rate index of | | standard |
| | can be obtained | the central point | | deviation, which |
| | from all points. | relative to the | | will be used as |
| | | best index | | input to a learning |
| | | measured in the | | system. |
| | | same session. | | |
| 2 + 3 | Measurement of | It will be shown | A change in the | Extracting |
| | the type of beats. | the shape of the | shape of the | features from the |
| | | pulse in time and | graph of the pulse | graph, for |
| | | frequency: | (width, slopes) | example average |
| | | Graph 1: x-axis is | may indicate a | slope and |
| | | session time, y- | stenosis. | standard |
| | | axis is amplitude | | deviation, which |
| | | Graph 2: x-axis is | | will be used as |
| | | frequency, y-axis | | input to a learning |
| | | is amplitude | | system. Things |
| | | | | that can be |
| | | | | correlative to an |
| | | | | “angry” pulse |
| | | | | versus a “calm” |
| | | | | pulse as |
| | | | | described by the |
| | | | | medical |
| | | | | caretakers in the |
| | | | | palpation |
| | | | | examination |

Exemplary additional information to be displayed:
LOOK

| | | | |
|---|---|---|---|
| RGB image of the | A collection of | Comparison | The redness of |
| hand, with | RGB images | between images | the fistula may be |
| maximum FOV | across sessions | throughout the | monitored. |
| and full ROI. | during the | follow-up period | |
| | tracking period of | in relation to the | |
| | an arm at rest | current image. | |
| | parallel to the | | |
| | floor. | | |

### Exemplary System Features:

It should be understood that the following features are exemplary features and that additional and/or alternative features are also included in the scope of the invention, for example, features related to prediction of stenosis with the help of ML and displaying the information by projecting a light pattern on the patient's arm.

### Exemplary Features in “Look”:

AVF grayscale Image-Two gray-scaled images, gathered from infrared laser illumination. The infrared illumination intensifies blood vessel features, comparing to human-visible spectrum.

The grayscale images will be taken during one session: before the arm-elevation test, where the arm is parallel to the floor, and after the arm-elevation test, where the arm is above the heart level, and a few seconds passed such that the AVF is not fast-changing.

The medical caretaker can navigate between the grayscale images before and after the arm-elevation test on two different screens, for example, and navigate between the screens by using the buttons in the main menu.

The medical caretaker can see and compare AVF grayscale images from previous sessions, performed on previous dates. The comparison may be performed by seeing the current session grayscale image in one figure, and a grayscale image from a previous session in another figure. The figures may be displayed side-by-side on a single screen, for example.

Alternatively, the grayscale images from the current session and a previous session may be displayed in one figure.

AVF 3D shape-3D contour images of the arm and the fistula, before and after the arm-elevation test. The 3D image can be freely rotated, and can also be specified to XY, XZ or YZ planes.

The 3D images from previous sessions can be compared, as described in the grayscale image.

### Exemplary Features in “Listen”:

Vibration spectrum: The graph of the vibration spectrum displays an FFT transform of the temporal vibration signal, as described in “Sound recording”. In an exemplary embodiment, the x-axis of the signal may be limited between 20 Hz to 1000 Hz. Spectrums from previous sessions may be viewed as well.

In some embodiments, additional measurements (listed hereinbelow) may be provided. Each measurement may be in a form of a plot, where the x axis is the session time, and the y axis is the corresponding value. Trends in the changing values may be observed. Optionally, the system may provide recommendations based on the measurements.

Such measurements can be, for example:
The frequency index with the maximum value, above minimum index such as 250 HZ. Incremental of the maximal frequency index may indicate stenosis.
The ratio between the maximal value, above minimal index such as 250 HZ, and the maximal value beneath minimal index, such as 50 Hz. In other words, normalize the energy of the high abnormal frequencies, against the energy in low common frequencies. An increase of this value can indicate stenosis.
The FWHM (the width corresponding to half of value), of the maximum frequency index, as described hereinabove. Decrement of this value may indicate stenosis.

Sound recording: The optical vibrometry is converted to audible format, and the vibration records, in the current session and in previous sessions, can be listened to by navigating between them.

### Exemplary Features in “Feel”:

Heart rate estimation from the current session and previous sessions may be compared on a graph, where the x-axis is the session date and the y-axis is the heart rate in beats-per-minute (bpm). The y axis is limited to human bpm values, such as 30 to 150 bpm.

### Exemplary Features at the Product Level

| | | | | Output of the system / |
|---|---|---|---|---|
| | Feature Name | | Measurement [units] | User interface |
| Look | AVF diameter | | Diameter [mm] | Options: |
| | | | Diameter change - graph | Number |
| | | | (dates / mm) | (measurement) |
| | AVF shape | | Sketch | Number (Score |
| | | | Overlay with previous | combining two or |
| | | | sketches / one next to | more features) |
| | | | other by date | Graph (x[ ],y[ ]) |
| | Arm swelling | | Width [mm] | Sound file |
| | | | Width change; graph | Grayscale image |
| | | | (dates / mm) | Sketch |
| | Arm elevation | | Volume [ml] | Schematic coloring |
| | test: | | Graph volume / date | to draw the |
| | 1. | AVF | Volume change rate | clinician's attention |
| | | volume | [ml/sec] | to potential AVF |
| | | before and | | dysfunction |
| | | after | | Attributes |
| | | elevation | | Note that |
| | 2. | AFV | | measurement is |
| | | volume | | above or below a |
| | | change rate | | threshold value |
| | | Aneurism | Three-dimensional | Visible projections |
| | | | surface features, such as | on the arm |
| | | | smoothness and/or | |
| | | | roughness | |
| | | | Attributes: | |
| | | | Aneurism yes / no / | |
| | | | enlargement | |
| | AVF grayscale | | Color | |
| | Image | | Color changes | |
| | | | Overlay with previous | |
| | | | Images by date | |
| | branching & | | Size width and length | |
| | size of | | [mm] | |
| | collateral veins | | Density [collateral veins / | |
| | | | [mm] | |
| Listen (at | Bruit | | Graph: Frequency / | |
| human audible | | | amplitude | |
| frequencies) | | | Overlay with previous | |
| | | | graphs by date | |
| | | | Attributes: | |
| | | | Normal | |
| | | | High-pitched | |
| | | | predominantly systolic | |
| | | | bruit | |
| | Sound | | Sound file | |
| | recording | | Reference sound files | |
| | | | (previous files from same | |
| | | | patient by date, normal | |
| | | | and abnormal general | |
| | | | references) | |
| Feel (extending | Thrill | | Graph: Frequency / | |
| to below human | | | amplitude | |
| audible | | | Overlay with previous | |
| frequencies) | | | graphs by date | |
| | | | Attributes: | |
| | | | Normal (continuous thrill), | |
| | | | presence of a | |
| | | | discontinuous/systolic-only | |
| | | | thrill, absence of a thrill | |

| | Pulsatility | Pulse rate (Pulse per minute) | |
|---|---|---|---|
| | | Attributes: Normal, hyper- | |
| | | pulsation, a water-hammer | |
| | | pulse, or an “angry” pulse | |
| Suggest | Mapping of | Information regarding | Suggested location for |
| locations for | skin vibrations | vibration on the patient's arm | inflow and outflow |
| inflow and | Mapping of | | needle insertion point |
| outflow needle | blood vessels | | Suggested needling plan |
| insertion points | Mapping of | | based on analyzing at |
| | pulsatility | | least two images |

captured at different
times
Displaying the information
by projecting a light pattern
on the patient's arm

Although particular implementations have been disclosed herein in detail, this has been done by way of example for purposes of illustration only, and is not intended to be limiting with respect to the scope of the claims which follow. In particular, it is contemplated that various substitutions, alterations, and modifications may be made without departing from the spirit and scope of the disclosure as defined by the claims. Other aspects, advantages, and modifications are considered to be within the scope of the following claims. The claims presented are representative of the implementations and features disclosed herein. Other unclaimed implementations and features are also contemplated. Accordingly, other implementations are within the scope of the following claims.

It is expected that during the life of a patent maturing from this application many relevant image processing algorithms will be developed and the scope of the term image processing is intended to include all such new technologies a priori.

As used herein with reference to quantity or value, the term “approximately” means “within ±15% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for displaying data on a patient's arm, the method comprising:
capturing an image of a patient's arm;
analyzing the image, thereby obtaining information regarding a blood vessel in the patient's arm;
displaying the information by projecting a light pattern on the patient's arm.

2. The method according to claim 1, wherein the projecting comprises projecting using a Digital Light Processing (DLP) projector.

3. The method according to claim 1, wherein the information comprises a mapping of skin vibrations on the patient's arm.

4. The method according to claim 1, wherein the information comprises a mapping of blood vessels in the patient's arm.

5. The method according to claim 1, wherein the information comprises a mapping of pulsatility in the patient's arm.

6. The method according to claim 1, wherein the information comprises a suggested location for inflow needle insertion point in the patient's arm.

7. The method according to claim 1, wherein the information comprises a suggested location for outflow needle insertion point in the patient's arm.

8. The method according to claim 1, wherein the information comprises a location where it is suggested not to insert a needle.

9. The method according to claim 1, wherein the information comprises instructions for treatment.

10. The method according to claim 1, wherein the information comprises numerical data produced as a result of the analysis.

11. The method according to claim 1, wherein the information comprises a needling plan.

12. The method according to claim 11, wherein the needling plan is based on analyzing more than one image, at least two of the images used for the analyzing captured at different times; and wherein the needling plan is based on historic information on previous insertion points to suggest locations for inflow and outflow needle insertion points.

13. The method according to claim 1, wherein:
the capturing the image of the patient's arm comprises capturing a plurality of images of the patient's arm, and
the analyzing the image comprises analyzing the plurality of images.

14. The method according to claim 1, further comprising displaying said captured image, including said information, on a display.

15. A system for displaying data on a patient's arm, the system comprising:
a light source for illuminating a patient's arm;
a sensor for capturing an image of the patient's arm;
a computer for analyzing the image, thereby obtaining information regarding blood vessel in the patient's arm;
a projector for displaying the information by projecting a light pattern on the patient's arm.

16. The system according to claim 15, wherein the light source comprises a laser illuminator arranged to illuminate an area on the patient's arm.

17. The system according to claim 15, wherein the light source comprises a laser illuminator arranged to illuminate an area on the patient's arm with a pattern of spots.

18. The system according to claim 15, wherein the light source and/or the projector comprise a source of structured light.

19. The system according to claim 15, wherein the projector comprises a Digital Light Processing (DLP) projector; and wherein the source of structured light comprises a same DLP projector as the projector.

20. The system according to claim 15, further comprising a display configured for displaying said captured image including said obtained information.
